(19) United States
(12) Patent Application Publication
(10) Pub. No.: US 2026/0338774 A1
(43) Pub. Date: Oct. 29, 2026

(54) METHODS OF DETECTING CITRULLINATED PROTEINS

(71) Applicant: Sonoma Biotherapeutics, Inc., South San Francisco, CA (US)

(72) Inventors: Sara CHARMSAZ, Seattle, WA (US); Michelle BLAKE, Seattle, WA (US); Jeffrey TRACY, Bainbridge Island, WA (US); Joshua BEILKE, Seattle, WA (US); Anne-Renee VAN DER VUURST DE VRIES, South San Francisco, CA (US); Kathryn HOOPER, Seattle, WA (US)

(73) Assignee: Sonoma Biotherapeutics, Inc., South San Francisco, CA (US)

(21) Appl. No.: 19/490,273

(22) PCT Filed: Jun. 6, 2024
(86) PCT No.: PCT/US2024/032778

§ 371 Date: Dec. 4, 2025

Related U.S. Application Data
(60) Provisional application No. 63/506,816, filed on Jun. 7, 2023.

(51) Int. Cl. G01N 33/564; A61K 40/11; A61K 40/31; A61K 40/41; G01N 33/58

(52) U.S. Cl. CPC G01N 33/564; A61K 40/11; A61K 40/31; A61K 40/416; G01N 33/581; G01N 2440/18; G01N 2800/52

(57) ABSTRACT

Disclosed herein are methods of detecting citrullinated polypeptides and/or citrullinated peptides. Citrullinated polypeptides, such as citrullinated vimentin, fibrinogen, and filaggrin, are expressed in various body fluids and/or tissues of individuals with autoimmune diseases, for example the synovium of subjects with rheumatoid arthritis. Further disclosed are methods of diagnosing, treating, selecting an individual for treatment of, and monitoring the treatment of autoimmune diseases, such as rheumatoid arthritis or hidradenitis suppurativa. Kits for use with the methods described herein are also provided.

### CROSS-REFERENCE TO RELATED APPLICATIONS

This application claims the benefit of, and priority to, U.S. Provisional Application 63/506,816, filed on Jun. 7, 2023, the contents of which are incorporated herein by reference in their entirety.

### REFERENCE TO AN ELECTRONIC SEQUENCE LISTING

The contents of the electronic sequence listing (237752001440seqlist.xml; Size: 49,414 bytes; and Date of Creation: Jun. 5, 2024) are herein incorporated by reference in their entirety.

### BACKGROUND

The immune system plays a critical role in maintaining organismal homeostasis, poised between the elimination of foreign antigens and the self-tolerance of autoantigens. In particular, hyperactive immune dysregulation can lead to various autoimmune or inflammatory diseases (e.g., rheumatoid arthritis, hidradenitis suppurativa, irritable bowel syndrome, systemic lupus erythematosus, alopecia areata, multiple sclerosis), which is often the result of overactive effector lymphocytes or myeloid cells (e.g., macrophages), or underactive regulatory T lymphocytes (i.e., Tregs). Current therapies for autoimmune or inflammatory diseases involve the administration of steroids, which can cause serious side-effects in patients and often offer little relief. Currently, the average time to diagnosis for autoimmune diseases is 4.6 years, which delays the onset of treatment during a critical period of time for slowing or preventing disease progression and the associated long-term organ damage.

Rheumatoid Arthritis (RA) is a chronic inflammatory disease that targets peripheral joints and leads to bone erosion, impairment of mobility, and decreased quality of life. RA affects approximately 0.5-1% of the population worldwide, and the incidence rate keeps rising. The pathogenesis of RA is mainly localized in the synovial joint, where immune cells composed of T cells, B cells, macrophages, and dendritic cells infiltrate the synovium. Synovial hyperplasia in RA results in the infiltration of the synovium by immune cells, and subsequent cartilage damage and bone erosion. Moreover, fibroblast-like synoviocytes that are present in the sub-lining layer of the synovium proliferate and contribute to cartilage damage.

Dysregulated citrullination is a key factor driving the production and maintenance of antibodies to citrullinated proteins. RA autoimmunity is often characterized by the generation of anti-citrullinated protein antibodies (ACPA). Presence of ACPA in established RA is associated with RA severity and at early developmental phases can be predictive of an individual's likeliness to progress to full-blown RA.

Currently, there is no cure for RA. Lifelong treatment is usually required for patients with RA, which in addition of being extremely expensive, may cause severe side effects in the long term such as infections. Recent evidence showed that application of aggressive inflammation-targeted therapy within the first months from the onset of symptoms could benefit RA patients by slowing the rate of long-term structural damage, demonstrating the importance of early diagnosis and treatment. However, current diagnostic assays can provide false negatives in up to 50% of patients, indicating the importance of developing new assays and new treatments for individuals suspected of having RA and for RA patients.

Hidradenitis suppurativa (HS), also known as acne inversa, is a chronic inflammatory skin condition estimated to occur in approximately 1% of the U.S. population. HS is characterized by painful lumps and lesions forming under the skin that heal slowly, recur, and can lead to scarring. The disease can cause pain, movement restrictions, odor, and drainage from affected areas as well as disfigurement. Existing therapies and surgical interventions are inadequate for long term disease management, and there is no laboratory test currently available to diagnose HS, indicating the importance of developing new assays and new treatments for individuals suspected of having HS and for HS patients.

### BRIEF SUMMARY OF THE INVENTION

In one aspect, there is provided a method of detecting the presence or amount of a citrullinated polypeptide or citrullinated peptide in a biological sample obtained from an individual comprising a) incubating the biological sample with a capture antibody that binds to the citrullinated polypeptide or the citrullinated peptide to form a complex comprising the capture antibody and the citrullinated polypeptide or citrullinated peptide; b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody, thereby detecting the presence or amount of the citrullinated polypeptide or citrullinated peptide.

In another aspect is provided a method of diagnosing an autoimmune or inflammatory disease (e.g., rheumatoid arthritis or hidradenitis suppurativa) in an individual comprising a) incubating a biological sample obtained from the individual with a capture antibody that binds to a citrullinated polypeptide or citrullinated peptide to form a complex comprising the capture antibody and the citrullinated polypeptide or citrullinated peptide; b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with the autoimmune or inflammatory disease (e.g., rheumatoid arthritis or hidradenitis suppurativa) based upon the presence or amount of the citrullinated polypeptide or citrullinated peptide.

In another aspect is provided a method of selecting an individual having an autoimmune or inflammatory disease (e.g., rheumatoid arthritis or hidradenitis suppurativa) for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level. In some embodiments, the method further comprises comparing the amount of the citrullinated polypeptide or citrullinated peptide in the biological sample to a reference level or reference sample. In some embodiments, the reference sample is a biological sample obtained from an individual not having an autoimmune or inflammatory disease (e.g., rheumatoid arthritis or hidradenitis suppurativa).

In another aspect is provided a method of treating an autoimmune or inflammatory disease (e.g., rheumatoid arthritis or hidradenitis suppurativa) in an individual comprising i) detecting a citrullinated polypeptide or citrullinated peptide in a biological sample obtained from an individual comprising a) incubating the biological sample with a capture antibody that binds to a citrullinated polypeptide or citrullinated peptide to form a complex comprising the capture antibody and the citrullinated polypeptide or citrullinated peptide, b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody, and c) detecting the complex comprising the capture antibody, the citrullinated polypeptide or the citrullinated peptide, and the detection antibody; and ii) administering to the individual an effective amount of a composition comprising a T cell comprising a chimeric antigen receptor (CAR) comprising an antigen binding domain that specifically binds to a citrullinated polypeptide or citrullinated peptide, if the biological sample comprises citrullinated polypeptides or citrullinated peptides or an amount of citrullinated polypeptides or citrullinated peptides that is above a threshold or a reference level.

In another aspect is provided a method of monitoring treatment of an autoimmune or inflammatory disease (e.g., rheumatoid arthritis or hidradenitis suppurativa) in an individual comprising a) detecting the presence or amount of a citrullinated polypeptide or citrullinated peptide in a biological sample obtained from the individual receiving treatment for the autoimmune or inflammatory disease (e.g., rheumatoid arthritis or hidradenitis suppurativa); and b) comparing the presence or amount of the citrullinated polypeptide or citrullinated peptide in the biological sample to a biological sample obtained from the individual prior to receiving treatment for the autoimmune or inflammatory disease (e.g., rheumatoid arthritis or hidradenitis suppurativa) or from an earlier time point during treatment, wherein a reduction in the amount of citrullinated polypeptides or citrullinated peptides in a biological sample compared to the amount of citrullinated polypeptides or citrullinated peptides in a biological sample prior to receiving treatment for the autoimmune or inflammatory disease (e.g., rheumatoid arthritis or hidradenitis suppurativa) or from an earlier time point during treatment is indicative of the effective treatment for the autoimmune or inflammatory disease (e.g., rheumatoid arthritis or hidradenitis suppurativa) in the individual.

In another aspect is provided a method of diagnosing an autoimmune or inflammatory disease (e.g., rheumatoid arthritis or hidradenitis suppurativa) in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with the autoimmune or inflammatory disease (e.g., rheumatoid arthritis or hidradenitis suppurativa) based upon the presence or amount of the citrullinated polypeptide or citrullinated peptide.

In another aspect is provided a method of treating an autoimmune or inflammatory disease (e.g., rheumatoid arthritis or hidradenitis suppurativa) in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; b) detecting the signal emitted by the CAR to determine the presence or amount of the citrullinated polypeptide or citrullinated peptide; and c) administering to the individual an effective amount of a composition comprising a T cell comprising a chimeric antigen receptor (CAR) comprising an antigen binding domain that specifically binds to a citrullinated polypeptide or citrullinated peptide if the biological sample comprises citrullinated polypeptides or citrullinated peptides or an amount of citrullinated polypeptides or citrullinated peptides that is above a threshold or a reference level.

In some embodiments, the detecting comprises a) incubating the biological sample with a capture antibody that binds to the citrullinated polypeptide or citrullinated peptide to form a complex comprising the capture antibody and the citrullinated polypeptide or citrullinated peptide; b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody.

In some embodiments, the detecting comprises: a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide.

In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurativa.

In some embodiments, the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, blood, blood plasma, serum, synovial fluid, and any combination thereof. In some embodiments, the biological sample is serum. In some embodiments, the biological sample is synovial fluid. In some embodiments, the biological sample is serum and synovial fluid, wherein the serum and the synovial fluid are from the same individual.

In some embodiments, the capture antibody is immobilized to a sample plate. In some embodiments, the capture antibody is immobilized to a bead.

In some embodiments, the detection antibody is conjugated to a label molecule. In some embodiments, the detection antibody is detected by the binding of a secondary antibody, wherein the secondary antibody is conjugated to a label molecule. In some embodiments, the secondary antibody binds to a constant region of the detection antibody. In some embodiments, the secondary antibody is an anti-IgM antibody or fragment thereof. In some embodiments, the label molecule is selected from the group consisting of a chemiluminescent enzyme, a fluorescent tag, and a radiolabeled tag. In some embodiments, the label molecule is a horseradish peroxidase enzyme or an alkaline phosphatase enzyme. In some embodiments, the horseradish peroxidase enzyme or alkaline phosphatase enzyme bind a substrate molecule, wherein the substrate molecule is converted into a detectable product. In some embodiments, the substrate molecule is colorimetric/chromogenic, fluorescent, chemifluorescent, or chemiluminescent.

In some embodiments, the capture antibody and the detection antibody bind to different epitopes of the citrullinated polypeptide or citrullinated peptide. In some embodiments, the capture antibody and/or the detection antibody binds to one or more of: citrullinated vimentin, citrullinated filaggrin, citrullinated fibrinogen, citrullinated tenascin C, and/or citrullinated fragments thereof. In some embodiments, the capture antibody and/or the detection antibody binds to two or more of citrullinated vimentin, citrullinated filaggrin, citrullinated fibrinogen, citrullinated tenascin C, and/or citrullinated fragments thereof.

In some embodiments, the capture antibody or the detection antibody is an anti-peptidyl-citrulline antibody. In some embodiments, the anti-peptidyl-citrulline antibody is the F95 antibody clone.

In some embodiments, the capture antibody or the detection antibody comprises a full-length antibody or an antibody fragment selected from the group consisting of a Fv, scFv, camelid nanobody, heavy chain only antibody, and aptamer. In some embodiments, the capture antibody or the detection antibody comprises a full-length antibody. In some embodiments, the capture antibody comprises an Fc domain.

In some embodiments, the capture antibody, or the detection antibody, and/or the CAR comprises a VH and a VL, wherein: (i) the VH comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and (ii) the VL comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6. In some embodiments, the capture antibody, the detection antibody, or the CAR comprises a VH comprising the amino acid sequence of SEQ ID NO:7 or 8 and/or a VL comprising the amino acid sequence of SEQ ID NO:9 or 10. In some embodiments, the VH comprises the amino acid sequence of SEQ ID NO:7 and the VL comprises the amino acid sequence of SEQ ID NO:10.

In some embodiments, the methods described herein further comprise: a) detecting the level of one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in a biological sample; b) comparing the level of the one or more proteins to a reference level; and c) selecting the individual as having an autoimmune or inflammatory disease (e.g., rheumatoid arthritis or hidradenitis suppurativa) when the level of the one or more proteins in the biological sample is above the reference level.

In some embodiments, the administering comprises: (a) isolating T cells from a biological sample obtained from the individual; (b) enriching the T cells for Tregs; (c) transfecting the enriched Treg cells with an expression vector comprising an expression control sequence operatively linked to a nucleotide sequence encoding a chimeric antigen receptor (CAR) comprising an antigen binding domain that binds to a citrullinated polypeptide or citrullinated peptide; (d) expanding the transfected Treg cells; and (e) administering a composition comprising the expanded Treg cells to the individual.

In some embodiments, the individual is a human.

In some embodiments, the composition comprising the expanded Treg is administered intravenously.

In some embodiments, the CAR comprises an optional signal peptide, a CAR antigen binding domain that specifically binds to a citrullinated polypeptide or citrullinated peptide, an optional hinge domain, a transmembrane domain, one or more optional co-stimulatory domains, and an intracellular signaling domain.

In some embodiments, the CAR antigen binding domain binds to a citrullinated peptide or citrullinated fragment thereof. In some embodiments, the CAR antigen binding domain binds to one or more of: citrullinated vimentin, citrullinated filaggrin, citrullinated fibrinogen, citrullinated tenascin C, or citrullinated fragments thereof.

In some embodiments, the CAR antigen binding domain comprises an antibody fragment selected from the group consisting of an Fv, scFv, camelid nanobody, heavy chain only antibody, or aptamer. In some embodiments, the CAR antigen binding domain comprises an scFv. In some embodiments, the CAR antigen binding domain comprises the same VH and VL as the capture antibody or detection antibody as described herein. In some embodiments, the antigen binding domain of the CAR comprises an scFV comprising a VH and a VL joined by a linker selected from the group consisting of SEQ ID NOs:11-13. In some embodiments, the CAR antigen binding domain comprises the amino acid sequence of any one of SEQ ID NOs:14-16.

In some embodiments, the intracellular signaling domain is derived from CD3-zeta or an Fc receptor. In some embodiments, the CAR comprises at least one co-stimulatory domain derived from the group consisting of: CD3-zeta, FceR1g, Fcg, CD28, 4-1BB, OX40, CD40L, MyD88, CD40, ICOS, RANK/TRANCE-R, CTLA-4, CTLA-4/CD-28 hybrid, DAP10, CD27, and 2B4. In some embodiments, the CAR further comprises a hinge domain.

In one aspect is provided a kit comprising a capture antibody and a detection antibody and instructions for use according to any one of the methods described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 provides a schematic overview of the antibody-based detection assay setup.

FIG. 2 shows a dot plot of the results from the antibody-based detection assay in un-matched serum and synovial fluid samples from patients with rheumatoid arthritis (RA) or healthy controls. The dashed line indicates the threshold for detecting the presence of citrullinated polypeptides. Colorimetric readout was performed on a standard colorimetric plate reader at OD450 nm. RA, rheumatoid arthritis; OD, optical density.

FIGS. 3A-3B show detection using a cell-based CAR-Jurkat reporter assay. FIG. 3A provides a schematic overview of the cell-based detection assay. FIG. 3B shows a dot plot of the results from the cell-based assay in synovial fluid samples from patients with rheumatoid arthritis (RA) or healthy controls. The dashed line indicates the threshold for detecting the presence of citrullinated polypeptides. The level of luminescence was reported as RLU. RA, rheumatoid arthritis; RLU, relative light unit.

FIG. 4 shows the correlation between the results from the antibody-based detection assay and the cell-based assay. Data from the cell-based assay is presented on the y-axis, and data from the antibody-based detection assay is presented on the x-axis. Each dot represents one patent sample. The percentage of patients with rheumatoid arthritis with detected levels of citrullinated polypeptides is also presented.

FIG. 5 shows the correlation between the results from the antibody-based detection assay testing citrullinated polypeptide levels in synovial fluid and serum. Data from serum samples is presented on the y-axis, and data from synovial fluid samples is presented on the x-axis. Each dot represents one patent sample. The percentage of patients with rheumatoid arthritis with detected levels of citrullinated polypeptides is also presented. Antibody-based, antibody-based detection assay; RA, rheumatoid arthritis; SD, standard deviation; SF, synovial fluid.

FIG. 6 provides a heatmap showing the correlational relationships between citrullinated polypeptide levels detected using the antibody-based detection assay in synovial fluid or serum and systemic markers of inflammation, including CRP, TNF-α, IL-1β, IL-6, IL-12p70, IL-18, IP-10, MCP-1, and MIP-1α. Blue squares indicate positive correlation, and red squares indicate negative correlation. The darker square color indicates a stronger correlation. Ab, antibody-based detection assay; CRP, C reactive protein; RLU, relative light units; SF, synovial fluid; OD, optical density.

FIGS. 7A-7B provide correlative analysis of the antibody-based detection assay and a CRP ELISA. FIG. 7A provides an overview summary of RA patient characteristics. FIG. 7B shows the correlation between the results from synovial fluid using the antibody-based detection assay and a CRP ELISA assay for serum samples. Data from the CRP ELISA on serum samples is presented on the y-axis, and data from antibody-based detection assay on synovial fluid samples is presented on the x-axis. Each dot represents one patent sample. A box plot providing the levels of CRP in patients who are positive or negative for the presence of citrullinated polypeptides is also provided. Antibody, antibody-based detection assay; CRP, C reactive protein; SF, synovial fluid; OD, optical density.

### DETAILED DESCRIPTION

### I. Definitions

Unless otherwise specified, terms and symbols of biochemistry, nucleic acid chemistry, molecular biology, developmental biology and molecular genetics follow those of standard treaties and texts in the field, for example, Sambrook et al, Molecular Cloning: A Laboratory Manual, 2nd Edition (Cold Spring Harbor Press, 1989); Alberts and Singer, Developmental Biology, Eighth Edition (Sinauer Associates Inc., Sunderland, MA, 2006); Kornberg and Baker, DNA Replication, Second Edition (W.H. Freeman, New York, 1992); Gaits, ed., Oligonucleotide Synthesis: A Practical Approach (IRL Press, Oxford, 1984); Lehninger, Biochemistry, Second Edition (Worth Publishers, New York, 1975); Eckstein, ed., Oligonucleotides and Analogs: A Practical Approach (Oxford University Press, New York, 1991); and the like.

As used herein, the terms “antigen,” “immunogen,” and “antibody target,” refer to a molecule, compound, or complex that is recognized by an antibody, i.e., can be bound by the antibody. The term can refer to any molecule that can be recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “antibody” refers to a polypeptide comprising a framework region from an immunoglobulin gene, that specifically bind and recognize an antigen. Typically, the “variable region” contains the antigen-binding region of the antibody (or its functional equivalent) and is most critical in specificity and affinity of binding. An antibody may be a full-length antibody, or an antibody fragment, such as a Fv, Fab, or scFv.

Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (e.g., IgG1, IgG2, IgG3, IgG4, IgA1 and IgA2). The light chains can be either lambda or kappa.

The following are a non-exhaustive list of different antibody forms, all retaining antigen binding activity:
(1) whole immunoglobulins (also referred to as “intact” antibodies) (two light chains and two heavy chains, e.g., a tetramer);
(2) an immunoglobulin polypeptide (a light chain or a heavy chain);
(3) an antibody fragment, such as Fv (a monovalent or bi-valent variable region fragment, and can encompass only the variable regions (e.g., V_{L} and/or V_{H}), Fab (V_{L}C_{L} V_{H}C_{H}), F(ab′)2, Fv (V_{L}V_{H}), scFv (single chain Fv) (a polypeptide comprising a V_{L} and V_{H} joined by a linker, e.g., a peptide linker), (scFv)2, sc(Fv)2, bispecific sc(Fv)2, bispecific (scFv)2, minibody (sc(FV)2 fused to CH3 domain), diabody (noncovalent dimer of single-chain Fv (scFv) fragment that consists of the heavy chain variable (VH) and light chain variable (VL) regions connected by a small peptide linker), triabody is trivalent sc(Fv)3 or trispecific sc(Fv)3;
(4) a multivalent antibody (an antibody comprising binding regions that bind two different epitopes or proteins, e.g., “scorpion” antibody;
(5) a fusion protein comprising a binding portion of an immunoglobulin fused to another amino acid sequence (such as a fluorescent protein); and
(6) heavy chain only antibody or antibody fragment having only two heavy chains and lacking the two light chains usually found in antibodies.

Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

The phrase “CDR sequence set” as used herein refers to the 3 heavy chain and/or 3 light chain CDRs of a particular antibody described herein. A “light chain” CDR sequence set refers to the light chain CDR sequences. A “heavy chain” CDR sequence set refers to the heavy chain CDR sequences. A “full” CDR sequence set refers to both heavy chain and light chain CDR sequences. CDRs are predicted based on IMGT sequence alignment.

As used herein, the term “chimeric antibody” refers to an antibody having amino acid sequences derived from two or more species. In one embodiment, the variable regions of both light and heavy chains correspond to the variable region of antibodies derived from one species of mammal (e.g., mouse, rat, rabbit, etc.) with the desired specificity, affinity and capability, while the constant regions are homologous the sequence derived from another species (typically in the subject receiving the therapy, e.g., human) to avoid eliciting an immune response.

As used herein, the term “humanized antibody” refers to a chimeric antibody in which the CDRs, obtained from the VH and VL regions of a non-human antibody having the desired specificity, affinity and capability are grafted to a human framework sequence. In one embodiment, the framework residues of the humanized antibody are modified to refine and optimize the antibody specificity, affinity and capability. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996).

As used herein, the term “human antibody” refers to an antibody produced by a human or an antibody having an amino acid sequence corresponding thereto made by any technique known in the art.

The specificity of the binding can be defined in terms of the comparative dissociation constants (Kd) of the antibody (or other targeting moiety) for target, as compared to the dissociation constant with respect to the antibody and other materials in the environment or unrelated molecules in general. A larger (higher) Kd is a Kd that describes a lower affinity interaction. Conversely a smaller (lower) Kd is a Kd that describes a higher affinity interaction or tighter binding. By way of example only, the Kd for an antibody specifically binding to a target may be femtomolar, picomolar, nanomolar, or micromolar and the Kd for the antibody binding to unrelated material may be millimolar or higher. Binding affinity can be in the micromolar range (kD=10⁻⁴ to 10⁻⁶), nanomole range (kD=10⁻⁷ M to 10⁻⁹ M), picomole range (kD=10⁻¹⁰ M to 10⁻¹² M), or femtomole range (kD=10⁻¹³ M to 10⁻¹⁵ M).

As used herein, an antibody “binds” or “recognizes” an antigen or epitope if it binds the antigen or epitope with a Kd of less than 10⁻⁴M (i.e., in the micromolar range). The term “binds” with respect to a cell type (e.g., an antibody that binds cancer cells), typically indicates that an agent binds a majority of the cells in a pure population of those cells. For example, an antibody that binds a given cell type typically binds to at least ⅔ of the cells in a population of the indicated cells (e.g., 67, 75, 80, 85, 90, 91, 92, 93, 94, 95, 96, 97, 98, 99, or 100%). In some cases, binding to a polypeptide can be assayed by comparing binding of the antibody to a cell that presents the polypeptide to binding (or lack thereof) of the antibody to a cell that does not express the polypeptide. One of skill will recognize that some variability will arise depending on the method and/or threshold of determining binding. Affinity of an antibody for a target can be determined according to methods known in the art, e.g., as reviewed in Ernst et al. Determination of Equilibrium Dissociation Constants, Therapeutic Monoclonal Antibodies (Wiley & Sons ed. 2009).

As used herein, the term “greater affinity” as used herein refers to a relative degree of antibody binding where an antibody X binds to target Y more strongly (Kon) and/or with a smaller dissociation constant (Koff) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as K_{A} equal to 1/K_{D} where K_{D} is equal to kₒₙ/k_{off}. The kₒₙ and k_{off} values can be measured using surface plasmon resonance technology, for example, using a Molecular Affinity Screening System (MASS-1) (Sierra Sensors GmbH, Hamburg, Germany). An antagonist or blocking antibody is an antibody that partially or fully blocks inhibits or neutralizes a biological activity related to the target antigen relative to the activity under similar physiological conditions when the antibody is not present. Antagonists can be competitive, non-competitive, or irreversible. A competitive antagonist is a substance that binds to a natural ligand or receptor at the same site as the natural ligand-receptor interaction or binds allosterically in a manner that induces a change to prevent normal binding. A non-competitive antagonist binds at a different site than the natural ligand-receptor interaction but lowers the K_{D} or signal resulting from the interaction. An irreversible inhibitor causes covalent modifications to the receptor preventing any subsequent binding.

As used herein, the term “avidity” refers to the overall stability of the binding complex between the antibody and the target antigen. It is governed by three factors, (i) the intrinsic affinity of the antibody for the antigen, (2) the valency of the antibody, and (3) the geometric arrangement of the interacting components. Affinity is the strength of the interaction between the antibody and a single target, whereas avidity is an accumulated strength of multiple affinities. In one embodiment, the antibodies provided herein are divalent.

As used herein, an antibody “preferentially binds” binds a first antigen relative to a second antigen if it binds the first antigen with greater affinity than it does the second antigen. Preferential binding can be at least any of 2-fold, 5-fold, 9-fold, 10-fold, 20-fold, 30-fold, 40-fold, 50-fold, 100-fold, 500-fold or 1000-fold greater affinity.

As used herein, an antibody “specifically binds” or is “specific for” a target antigen or target group of antigens if it binds the target antigen or each member of the target group of antigens with an affinity of at least any of 1×10⁻⁶ M, 1×10⁻⁷M, 1×10⁻⁸ M, 1×10⁻⁹ M, 1×10⁻¹⁰ M, 1×10¹¹ M, 1×10⁻¹² M, and, for example, binds to the target antigen or each member of the target group of antigens with an affinity that is at least two-fold greater than its affinity for non-target antigens to which it is being compared. Typically, specific binding is characterized by binding the antigen with sufficient affinity that the antibody is useful as a diagnostic to detect the antigen or epitope and/or as a therapeutic agent in targeting the antigen or epitope.

As used herein, an antibody “blocks” or “antagonizes” the binding of a ligand to receptor when it competitively reduces or prevents interaction all of the ligand with the receptor. In an embodiment, the measured level of reduction can be at least any of 5%, 10%, 25%, 50%, 80%, 90%, 95%, 97.5%, 99%, 99.5%, 99.9% of a control (e.g., untreated) cell.

As used herein, the term “polypeptide” refers to a molecule having a sequence of natural and/or unnatural amino acids connected through peptide bonds. The term “peptide” refers to a short polypeptide, typically no more than 30 amino acids long. The amino acid sequence of a polypeptide is referred to as its “primary structure.” The term “protein” refers to a polypeptide having a secondary, tertiary and/or quaternary structure, e.g., structures stabilized by hydrogen bonds, relationships between secondary structures and structures formed of more than one protein. Proteins can be further modified by other attached moieties such as carbohydrate (glycoproteins), lipids (lipoproteins) phosphate groups (phosphoproteins) and the like.

As used herein, an amino acid sequence “consists of” only the amino acids in that sequence.

As used herein, a first amino acid sequence “consists essentially of” a second amino acid sequence if the first amino acid sequence (1) comprises the second amino sequence and (2) is no more than 1, no more than 2 or no more than 3 amino acids longer than the second amino acid sequence.

As used herein, a first amino acid sequence is a “fragment” of a second amino acid sequence if the second amino acid sequence comprises the first amino acid sequence. In certain embodiments, a first amino acid sequence that is a fragment of a second amino acid sequence may have no more than any of 1, 2, 3, 4, 5, 6, 7, 8, 9 or 10 fewer amino acids than the second amino acid sequence.

As used herein, a “functional equivalent” of a reference amino acid sequence is a sequence that is not identical to the reference sequence, but that contains minor alterations such as, for example, insertion, deletion, or substitution of one or a few amino acids. A functionally equivalent sequence retains the function (e.g., immunogenicity) of the reference sequence to which it is equivalent. If a functionally equivalent amino acid sequence contains substitution of one or more amino acids with respect to the reference sequence, these will generally be conservative amino acid substitutions.

As used herein, a “conservative amino acid substitution” is one in which one amino acid residue is replaced with another amino acid residue without abolishing the protein's desired properties. Suitable conservative amino acid substitutions can be made by substituting amino acids with similar hydrophobicity, polarity, and R-chain length for one another. See, e.g., Watson, et al., “Molecular Biology of the Gene,” 4th Edition, 1987, The Benjamin/Cummings Pub. Co., Menlo Park, CA, p. 224. Examples of conservative amino acid substitution include the following (Note, some categories are not mutually exclusive):

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic (non-polar, | Ala, Val, Ile, Leu, Met, Gly, Pro |
| hydrophobic) | |
| Basic | Lys, Arg, His |
| Aromatic | Phe, Tyr, Trp |

As used herein, the term “substantially identical” refers to identity between a first amino acid sequence that contains a sufficient or minimum number of amino acid residues that are i) identical to, or ii) conservative substitutions of aligned amino acid residues in a second amino acid sequence such that the first and second amino acid sequences have a common structural domain and/or common functional activity and/or common immunogenicity. For example, amino acid sequences that contain a common structural or antigenic domain having at least about 90%, 91%, 92%, 93%, 94%, 95%, 96%, 97%, 98% or 99% identity are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, or encode polypeptides having the same immunogenic properties.

As used herein, a chemical entity, such as a polypeptide, is “substantially pure” or “isolated” if it is the predominant chemical entity of its kind (e.g., of polypeptides) in a composition. This includes the chemical entity representing more than 50%, more than 80%, more than 90%, more than 95%, more than 98%, more than 99%, more than 99.5%, more than 99.9%, or more than 99.99% of the chemical entities of its kind in the composition. A substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules, stabilizers (e.g., BSA), and elemental ion species are not considered macromolecular species for purposes of this definition.

The phrase “isolated antibody” refers to antibody produced in vivo or in vitro that has been removed from the source that produced the antibody, for example, an animal, hybridoma or other cell line (such as recombinant insect, yeast or bacterial cells that produce antibody).

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules of the present application. BLAST protein searches can be performed with the XBLAST program parameters set, e.g., to score-50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, multiplied by 100 to convert to percentage.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. It is understood that polynucleotides comprising non-transcribable nucleotide bases may be useful as probes in, for example, hybridization assays. The nucleic acid can be either double stranded or single stranded, and represents the sense or antisense strand. Further, the term “nucleic acid” includes the complementary nucleic acid sequences as well as codon optimized or synonymous codon equivalents.

The term “isolated nucleic acid” as used herein refers to a nucleic acid substantially free of cellular material or culture medium when produced by recombinant DNA techniques, or chemical precursors, or other chemicals when chemically synthesized. An isolated nucleic acid is also substantially free of sequences that naturally flank the nucleic acid (i.e., sequences located at the 5′ and 3′ ends of the nucleic acid) from which the nucleic acid is derived.

As used herein, the term “expression construct” refers to a polynucleotide comprising an expression control sequence operatively linked with a heterologous nucleotide sequence (i.e., a sequence to which the expression control sequence is not normally connected to in nature) that is to be the subject of expression. As used herein, the term “expression vector” refers to a polynucleotide comprising an expression construct and sequences sufficient for replication in a host cell or insertion into a host chromosome. Plasmids and viruses are examples of expression vectors. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences).

As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, lentiviral vectors, Adeno Associated viral vectors and the like. The term “plasmid” as used herein generally refers to a construct of extrachromosomal genetic material, usually a circular DNA duplex, which can replicate independently of chromosomal DNA.

“Transfection” refers to the introduction of new genetic material into a cell. It includes transformation (the direct uptake and incorporation of exogenous genetic material from its surroundings through the cell membrane), transduction (the introduction of foreign DNA by a bacteriophage virus into a host cell) and conjugation.

As used herein, a “host cell” refers to a recombinant cell comprising an expression construct.

As used herein, “autoimmune disease” refers to a disease characterized by immune activity that targets an individual's healthy cells, tissues, and/or organs. An autoimmune disease may be characterized by overactive immune cells, for example by overactive macrophages, eosinophils, mast cells, effector T cells, etc. In some instances, regulatory immune cells (e.g., regulatory T cells (Tregs)) that provide inhibitory signals to immune cells (e.g., effector T cells) may be underactive or otherwise unable to effectively inhibit effector immune cells. As a result, autoimmune diseases are often accompanied by inflammation (e.g., inflammation localized within the affected organ system and/or systemic inflammation). In some instances, an individual suffering from one autoimmune disease may have an increased risk of developing one or more additional autoimmune diseases, for example type 1 diabetes and celiac disease wherein the two common HLA haplotypes (i.e., DR4-DQ8 and DR3-DQ2) lead to increased risk of developing both of these autoimmune diseases.

As used herein, “inflammatory disease” refers to a disease in which the level of inflammation is pathologically high and triggers disease progression, often by recruiting immune cells to the inflamed tissue or organ. Non-limiting examples of inflammatory diseases include allergy, asthma, autoimmune diseases, coeliac disease, glomerulonephritis, hepatitis, inflammatory bowel disease, preperfusion injury, and transplant rejection.

As used herein, the term terms “therapy,” “treatment,” “therapeutic intervention” and “amelioration” refer to any activity resulting in a reduction in the severity of symptoms. The terms “treat” and “prevent” are not intended to be absolute terms. Treatment and prevention can refer to any delay in onset, amelioration of symptoms, improvement in patient survival, increase in survival time or rate, etc. Treatment and prevention can be complete or partial. The effect of treatment can be compared to an individual or pool of individuals not receiving the treatment, or to the same patient prior to treatment or at a different time during treatment “Treating” and “Treatment” can also mean prolonging survival as compared to expected survival if not receiving treatment. “Treating” and “treatment” as used herein also include prophylactic treatment.

Compositions or methods “comprising” or “including” one or more recited elements may include other elements not specifically recited. For example, a composition that “comprises” or “includes” an antibody may contain the antibody alone or in combination with other ingredients.

As used herein, the term “individual” refers to a mammal, such as a human.

As used herein, the terms “effective amount,” “effective dose,” and “therapeutically effective amount,” refer to an amount of an agent that is sufficient to generate a desired response, such as reduce or eliminate a sign or symptom of a condition or ameliorate a disorder. In some examples, an “effective amount” is one that treats (including prophylaxis) one or more symptoms and/or underlying causes of any of a disorder or disease and/or prevents progression of a disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

### II. Methods of Detecting Citrullinated Polypeptides

Provided herein is a method of detecting the presence of the amount of a citrullinated polypeptide or citrullinated peptide, for example a citrullinated protein or fragment thereof, in a biological sample obtained from an individual.

In some embodiments, the method of detection comprises a) incubating the biological sample with a capture antibody that binds to the citrullinated polypeptide or the citrullinated peptide to form a complex comprising the capture antibody and the citrullinated polypeptide or citrullinated peptide; b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody, thereby detecting the presence or amount of the citrullinated polypeptide or citrullinated peptide.

In other embodiments, the method of detection comprises a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with an autoimmune or inflammatory disease (e.g., rheumatoid arthritis or hidradenitis suppurativa) based upon the presence or amount of the citrullinated polypeptide or citrullinated peptide. In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurativa.

Biological samples can be obtained from an individual (e.g., a human individual) from any one or more of cerebrospinal fluid, spinal cord white matter, lesional skin biopsy, sweat, saliva, pus, mucus, bile, liver biopsy, brain biopsy, pleural fluid, peritoneal fluid, blood, blood plasma, synovial fluid, serum, or any combination thereof. Methods of obtaining any of these biological samples, for example methods of obtaining synovial fluid and methods of obtaining serum, from a human individual are known in the art. In brief, synovial fluid can be extracted from the joint synovium in an individual, for example, by therapeutic arthrocentesis with aseptic procedure. Synovial fluids can be aspirated from affected joints (e.g., knee, knuckles) after acupuncture using a 30-mL syringe with 21-gauge needle. The isolated synovial fluid can then be centrifuged to generate the sample that can be used for any of the methods described herein or stored for future use (see, e.g., Ji et al., Front Cell Dev Biol. 2021; 9: 700879, hereby incorporated by reference in its entirety). Serum can be collected by harvesting whole blood from an individual, allowing the blood to clot at room temperature, and removing the clot by centrifugation, leaving the serum as the supernatant which can be used for any of the methods described herein or stored for future use. In some embodiments, synovial fluid is obtained from an individual. In some embodiments, serum is obtained from an individual. In some embodiments, individual-matched synovial fluid and serum are obtained from an individual. In some embodiments, the individual is human. In some embodiments, the individual is suspected of having, is diagnosed with having, or has an autoimmune or inflammatory disease (e.g., rheumatoid arthritis or hidradenitis suppurativa). In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis. In some embodiments, the autoimmune or inflammatory disease is hidradenitis suppurativa.

In some embodiments, the method of detection comprises a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with rheumatoid arthritis based upon the presence or amount of the citrullinated polypeptide or citrullinated peptide. In some embodiments, the biological sample is serum and/or synovial fluid. In some embodiments, the individual is suspected of having, is diagnosed with having, or has rheumatoid arthritis.

In some embodiments, the method of detection comprises a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with hidradenitis suppurativa based upon the presence or amount of the citrullinated polypeptide or citrullinated peptide. In some embodiments, the biological sample is serum, pus, and/or lesional skin biopsy. In some embodiments, the individual is suspected of having, is diagnosed with having, or has hidradenitis suppurativa.

### A. Citrullinated Polypeptides/Peptides

A known hallmark of various autoimmune or inflammatory diseases, in particular rheumatoid arthritis (RA), is the dysregulated citrullination profile of various proteins, polypeptides, peptides, and/or fragments thereof. Synovial fluid (SF) from individuals with RA contains a unique pattern of citrullination that includes proteins spanning the range of molecular weights, termed hypercitrullination. More than 100 citrullinated proteins have been identified by proteomic analysis from the cellular and soluble components of synovial fluid from individuals with RA, of which only a few have yet been identified as targets of autoantibodies (e.g., vimentin, α-enolase, and fibrinogen). Production of autoantibodies targeting hypercitrullinated polypeptides, for example anti-citrullinated protein antibodies, are correlated to RA disease severity. Protein citrullination (also known as deamination) occurs through the actions of the enzymes peptidylarginine deiminase (PAD) 1-4 and 6 in humans, wherein PADs convert sidechain peptidylarginine to peptidylcitrulline in a calcium-dependent, irreversible process. Citrullinated proteins, polypeptides, peptides, and/or fragments thereof that act as autoantigens can be generated in the synovium of an affected joint by resident fibroblast-like synoviocytes (FLSs), neutrophils, and monocytes, triggering an autoimmune reaction by B cells and T cells. See, e.g., Darrah, E. and Andrade, F., Curr Opin Rheumatol. 2018 January; 30(1): 72-78, hereby incorporated by reference in its entirety.

Additional autoimmune or inflammatory diseases that are associated with elevated levels of citrullinated peptides include, e.g., hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. For example, citrullinated peptides and anti-citrullinated autoantibodies were elevated in hidradenitis suppurativa sera and lesional skin compared to control subjects lacking hidradenitis suppurativa (Carmona-Rivera et al. (2022), J Invest Dermatol. 142(3): 924-935, hereby incorporated by reference in its entirety). Hypercitrullinated myelin basic protein (MBP) has been shown to elicit an immune response in patients with multiple sclerosis (see, e.g., Monreal et al. (2023), J. Autoimmun. 139:103092, hereby incorporated by reference in its entirety), and has been found to localize to regions of ongoing demyelination in MS lesions. In Alzheimer's disease patients, citrullination of Aβ in brain samples of subjects with sporadic AD and subjects with familial AD has been identified (see, e.g., Mukherjee et al. (2021), ACS Chem Neurosci.12(19):3719-3732, hereby incorporated by reference in its entirety), as well as the citrullination of Tau proteins (see, e.g., Shi et al. (2024), BioRXIV doi: https://doi.org/10.1101/2024.02.13.579999, hereby incorporated by reference in its entirety). Even with regard to periodontitis (e.g., aggressive periodontitis or chronic apical periodontitis), citrullination has been demonstrated to contribute to the inflammatory pathogenesis and lead to the production of anti-citrullinated autoantibodies (ACPA-s) in periodontitis patients (see, e.g., Martos et al. (2023), Int. Endod. J. 56(5):584-592, hereby incorporated by reference in its entirety).

In some embodiments, citrullinated polypeptides or citrullinated peptides are about any of at least about 3 amino acids long, about 4 amino acids long, about 5 amino acids long, about 6 amino acids long, about 7 amino acids long, about 8 amino acids long, about 9 amino acids long, about 10 amino acids long, about 11 amino acids long, about 12 amino acids long, about 13 amino acids long, about 14 amino acids long, about 15 amino acids long, about 16 amino acids long, about 17 amino acids long, about 18 amino acids long, about 19 amino acids long, about 20 amino acids long, about 25 amino acids long, about 30 amino acids long, about 35 amino acids long, about 40 amino acids long, about 45 amino acids long, about 50 amino acids long, about 55 amino acids long, about 60 amino acids long, about 65 amino acids long, about 70 amino acids long, about 75 amino acids long, about 80 amino acids long, about 85 amino acids long, about 90 amino acids long, about 95 amino acids long, about 100 amino acids long or more. In some embodiments, citrullinated polypeptides or citrullinated peptides are between about any of about 3 to about 7 amino acids in length, about 5 to about 10 amino acids in length, about 7 to about 13 amino acids in length, about 10 to about 15 amino acids in length, about 13 to about 17 amino acids in length, about 15 to about 20 amino acids in length, about 17 to about 23 amino acids in length, about 20 to about 25 amino acids in length, about 23 to about 27 amino acids in length, about 25 to about 30 amino acids in length, about 27 to about 33 amino acids in length, about 30 to about 35 amino acids in length, about 33 to about 37 amino acids in length, about 35 to about 40 amino acids in length, about 37 to about 43 amino acids in length, about 40 to about 45 amino acids in length, about 43 to about 47 amino acids in length, about 45 to about 50 amino acids in length, about 47 to about 53 amino acids in length, about 50 to about 55 amino acids in length, 53 to about 57 amino acids in length, about 55 to about 60 amino acids in length, about 57 to about 63 amino acids in length, about 60 to about 65 amino acids in length, about 63 to about 67 amino acids in length, about 65 to about 70 amino acids in length, about 67 to about 73 amino acids in length, about 70 to about 75 amino acids in length, about 73 to about 77 amino acids in length, about 75 to about 80 amino acids in length, about 77 to about 83 amino acids in length, about 80 to about 85 amino acids in length, about 83 to about 87 amino acids in length, about 85 to about 90 amino acids in length, about 87 to about 93 amino acids in length, about 90 to about 95 amino acids in length, about 93 to about 97 amino acids in length, about 95 to about 100 amino acids in length, about 97 to about 103 amino acids in length, about 100 to about 105 amino acids in length, or more.

In some embodiments, the citrullinated polypeptides and/or citrullinated peptides comprise protein fragments. In some embodiments, citrullinated polypeptides and/or citrullinated peptides comprise full-length proteins. In some embodiments, citrullinated polypeptides and/or citrullinated peptides comprise a combination of protein fragments and full-length proteins. In some embodiments, the citrullinated polypeptides and/or citrullinated peptides all comprise the same number of amino acids. In some embodiments, the citrullinated polypeptides and/or citrullinated peptides comprise different numbers of amino acids. In some embodiments, the citrullinated polypeptides and/or citrullinated peptides comprise a combination of polypeptides and/or peptides with the same number of amino acids and different number of amino acids.

In some embodiments, the citrullinated polypeptides and/or citrullinated peptides derive from the same protein, polypeptide, and/or peptide. In some embodiments, the citrullinated polypeptides and/or citrullinated peptides derive from different proteins, polypeptides, and/or peptides. For example, in some embodiments, the citrullinated polypeptides and/or citrullinated peptides comprise any one or more of: citrullinated vimentin, citrullinated filaggrin, citrullinated fibrinogen, citrullinated tenascin C, and/or citrullinated fragments thereof, including any two or more, three or more, four or more, or all five of citrullinated vimentin, citrullinated filaggrin, citrullinated fibrinogen, citrullinated tenascin C, and/or citrullinated fragments thereof.

### B. Antibody-Based Detection Assay

In one aspect is provided a method of detecting the presence of the amount of a citrullinated polypeptide or citrullinated peptide, for example a citrullinated protein or fragment thereof, in a biological sample obtained from an individual comprising a) incubating the biological sample with a capture antibody that binds to the citrullinated polypeptide or the citrullinated peptide to form a complex comprising the capture antibody and the citrullinated polypeptide or citrullinated peptide; b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody, thereby detecting the presence or amount of the citrullinated polypeptide or citrullinated peptide.

In some embodiments, the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, synovial fluid, whole blood, serum, plasma, and any combination thereof. In some embodiments, the biological sample is serum and/or synovial fluid. In some embodiments, the biological sample is serum. In some embodiments, the biological sample is synovial fluid. In some embodiments, the serum and the synovial fluid are from the same individual. In some embodiments, the individual is a human. In some embodiments, the individual has an autoimmune or inflammatory disease selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the individual has rheumatoid arthritis or hidradenitis suppurativa.

In some embodiments, the capture antibody is immobilized to a solid phase made from materials such as polymer, metal (paramagnetic, ferromagnetic particles), glass, and ceramic. In some embodiments, the solid phase is made from gel substances such as silica, alumina, and polymer gels. In some embodiments, the solid phase is made from zeolites and other porous substances. In some embodiments, the solid phase is a stationary component including, but not limited to, a microtiter plate, solid strip, cuvette, tube, or any suitable spectrometer sample container. In some embodiments, the solid phase is a non-stationary component including, but not limited to, beads and microparticles. Such non-stationary components include polymer particles or beads such as polystyrene and poly(methylmethacrylate); gold particles or beads such as gold nanoparticles and gold colloids; and ceramic particles or beads such as silica, glass, and metal oxide particles. In some embodiments, the non-stationary component is magnetic. In some embodiments, the capture antibody is immobilized to a sample plate (e.g., sample wells of a microtiter plate). In some embodiments, the capture antibody is immobilized to a bead.

In some embodiments, the capture antibody is immobilized to a solid phase by passive adsorption. In some embodiments, the capture antibody is immobilized to a solid phase via a specific binding pair including, but not limited to, streptavidin or avidin/biotin, antibody/antigen, lectin/polysaccharide, steroid/steroid binding protein, hormone/hormone receptor, enzyme/substrate, IgG/Protein A and/or G, etc. In such embodiments, a first component of the binding pair (e.g., streptavidin or avidin) is immobilized to the solid phase, and a second component of the binding pair (e.g., biotin) is conjugated to the capture antibody, wherein binding of the first and second component enables immobilization of the capture antibody to the solid phase. In some embodiments, the capture antibody can be covalently immobilized to a solid phase. In such embodiments, a reactive moiety on the solid phase can form a covalent bond with amino groups (lysins, alpha-amino groups), thiol groups (cystins, cysteines, and methionins), carboxylic acid groups (aspartic acids, glutamic acids), and sugar-alcoholic groups on the capture antibody. In some embodiments, the reactive moiety is an N-hydroxy-succinimide (NHS) ester, acid anhydride (e.g., maleic anhydride), maleimide, etc. In some embodiments, the solid phase with the immobilized capture antibody is treated with a blocking agent (e.g., bovine serum albumin, glycoprotein, polysorbates, etc.) to minimize non-specific binding of the capture antibody.

In some embodiments, the detection antibody is conjugated to a label molecule. In some embodiments, the detection antibody is detected by the binding of a secondary antibody, wherein the secondary antibody is conjugated to a label molecule. Label molecules can include, but are not limited to, any one of a chemiluminescent enzyme, a fluorescent tag, and a radiolabeled tag. For example, chemiluminescent enzymes commonly include a horseradish peroxidase enzyme or an alkaline phosphatase enzyme. The horseradish peroxidase enzyme or the alkaline phosphatase enzyme bind to a substrate molecule that gets converted into a detectable product. This substrate molecule can be any one of (e.g.) a colorimetric/chromogenic (e.g., tetramethylbenzidine (TMB) or p-nitrophenyl phosphate (pNPP)), a fluorescent, a chemifluorescent, or a chemiluminescent molecule. In some embodiments, the secondary antibody binds to a constant region of the detection antibody. In some embodiments, the secondary antibody IgA, IgD, IgE, IgG, or IgM antibody or fragment thereof. In some embodiments, the secondary antibody is an anti-IgM antibody or fragment thereof.

In some embodiments, the capture antibody, the detection antibody, and/or the secondary antibody can comprise an antibody fragment selected from the group consisting of a Fv, scFv, camelid nanobody, heavy chain only antibody, and aptamer. In some embodiments, the capture antibody, the detection antibody, and/or the secondary antibody can comprise an Fc domain.

In some embodiments, the capture antibody is the antibody described below and the detection antibody is an anti-peptidyl-citrulline antibody. In other embodiments, the capture antibody is an anti-peptidyl-citrulline antibody and the detection antibody is the antibody described below. In some embodiments, the capture antibody and the detection antibody bind to different epitopes of citrullinated polypeptides or citrullinated peptides. Both antibodies can bind to one or more of citrullinated vimentin, citrullinated filaggrin, citrullinated fibrinogen, citrullinated tenascin C, and/or citrullinated fragments thereof. For example, in some embodiments, the capture antibody and/or the detection antibody binds to one or more of: citrullinated vimentin, citrullinated filaggrin, citrullinated fibrinogen, citrullinated tenascin C, and/or citrullinated fragments thereof. In some embodiments, the capture antibody and/or the detection antibody binds to two or more of: citrullinated vimentin, citrullinated filaggrin, citrullinated fibrinogen, citrullinated tenascin C, and/or citrullinated fragments thereof. In some embodiments, the capture antibody and/or the detection antibody binds to three or more of: citrullinated vimentin, citrullinated filaggrin, citrullinated fibrinogen, citrullinated tenascin C, and/or citrullinated fragments thereof. In some embodiments, the capture antibody and/or the detection antibody binds to four or more of: citrullinated vimentin, citrullinated filaggrin, citrullinated fibrinogen, citrullinated tenascin C, and/or citrullinated fragments thereof. In some embodiments, the capture antibody and/or the detection antibody binds to each of: citrullinated vimentin, citrullinated filaggrin, citrullinated fibrinogen, citrullinated tenascin C, and citrullinated fragments thereof.

### 1. Antigen Binding Domain

### a) Structure

The antigen binding domain can include any polypeptide comprising an antigen binding function, e.g., an antibody as defined herein. In one embodiment, the antigen binding domain can comprise a heavy chain and a light chain. The heavy chain and/or the light chain can be connected to an Fc domain via a flexible linker.

### b) Targets

The antibody disclosed herein comprises an antigen binding domain that binds to citrullinated antigens, e.g., those found in the synovium of subjects with rheumatoid arthritis, the skin lesions of subjects with hidradenitis suppurativa, the myelin sheath or spinal cord white matter of subjects with multiple sclerosis, etc. In particular, the antigen binding domain can bind to one or more of (i) citrullinated vimentin, (ii) citrullinated filaggrin, (iii) citrullinated fibrinogen, (iv) tenascin C, and (v) citrullinated peptides thereof. In some embodiments, the antigen binding domain can bind to a citrullinated peptide fragment of (i)-(iv), wherein the peptide fragment is of at least about 10 amino acids in length or more, e.g., of at least about 12 amino acids, at least about 14 amino acids, at least about 16 amino acids in length, or more. In some embodiments, the antigen binding domain can bind to two or more of (i) citrullinated vimentin, (ii) citrullinated filaggrin, (iii) citrullinated fibrinogen, (iv) tenascin C, and (v) citrullinated peptide fragments thereof. In some embodiments, the antigen domain is one or more citrullinated peptides selected from the following sequences:

(SEQ ID NO: 27)
ST(Cit)SVSSSSY(Cit)(Cit)MFGG
(SEQ ID NO: 28)
VYAT(Cit)SSAV(Cit)L(Cit)SSV
(SEQ ID NO: 29)
(Cit)PAPPPISGGGY(Cit)A(Cit)
(SEQ ID NO: 30)
SHQEST(Cit)GRSRGRSGRSGS

The antigen binding domain can comprise sequences from an antibody VH and VL domain. This includes particular CDR sets from VH and VL domains. In some embodiments the antigen binding domain comprises the CDRs from the VH domains, wherein VH-CDR1 comprises the amino acid sequence of SEQ ID NO:1, VH-CDR2 comprises the amino acid sequence of SEQ ID NO:2, and VH-CDR3 comprises the amino acid sequence of SEQ ID NO:3. In some embodiments the target binding domain comprises the CDRs from the VL domains, wherein VL-CDR1 comprises the amino acid sequence of SEQ ID NO:4, VL-CDR2 comprises the amino acid sequence of SEQ ID NO:5, and VL-CDR3 comprises the amino acid sequence of SEQ ID NO:6. In some embodiments the antigen binding domain comprises the CDRs from VH and VL domains of SEQ ID NO:7 and SEQ ID NO:9, respectively. In some embodiments the target binding domain comprises the CDRs from VH and VL domains of SEQ ID NO:8 and SEQ ID NO: 10, respectively. In some embodiments the antigen binding domain comprises the CDRs from VH and VL domains of SEQ ID NO:7 and SEQ ID NO: 10, respectively. In some embodiments the antigen binding domain comprises the CDRs from VH and VL domains of SEQ ID NO:8 and SEQ ID NO:9, respectively.

In some embodiments the antigen binding domain comprises the complementarity-determining regions (CDRs) from VH and VL domains of SEQ ID NO:7 and SEQ ID NO: 10. In some embodiments, the VH domain of the antigen binding domain comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and the VL domain of the antigen binding domain comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6.

| | SEQ | | | |
|---|---|---|---|---|
| Region | ID | Sequence Fragment | Residues | Length |
| HFR1 | NO: 31 | HLHLQESGPGLVKPSETLSLTCTVSGGSIN | 1-30 | 30 |
| CDR- | NO: 1 | DTTYYWG | 31-37 | 7 |
| H1 | | | | |
| HFR2 | NO: 32 | WIRQPPGKGLEWIG | 38-51 | 14 |
| CDR- | NO: 2 | SIYYRGNTHYNSSLRS | 52-67 | 16 |
| H2 | | | | |
| HFR3 | NO: 33 | RVTMSVDTSKNRFSLKVTSVTAADTAVYYCA | 68-99 | 32 |
| | | R | | |
| CDR- | NO: 3 | LDPFDY | 100-105 | 6 |
| H3 | | | | |
| HER4 | NO: 34 | WGRGTLVTVSS | 106-116 | 11 |
| LFR1 | NO: 35 | SYVLTQPPSVSVAPGKTARITC | 1-22 | 22 |
| CDR- | NO: 4 | GGNNIGSKSVH | 23-33 | 11 |
| L1 | | | | |
| LFR2 | NO: 36 | WYQQKPGQAPVLVIY | 34-48 | 15 |
| CDR- | NO: 5 | YDSDRPS | 49-55 | 7 |
| L2 | | | | |
| LFR3 | NO: 37 | GIPERFSGSNSGNTATLTISRVEAGDEADYYC | 56-87 | 32 |
| CDR- | NO: 6 | QVWDSSSDHQV | 88-98 | 11 |
| L3 | | | | |
| LFR4 | NO: 38 | FGTGTKVTV | 99-108 | 11 |

In certain embodiments, the antigen binding domain comprises VH sequences selected from:
(1) VH #1
   HLHLQESGPGLVKPSETLSLTCTVSGGSINDTTYYWGWIRQPPGKGLE WIGSIYYRGNTHYNSSLRSRVTMSVDTSKNRFSLKVTSVTAADTAVY YCARLDPFDYWGRGTLVTVSS (SEQ ID NO:7; corresponding exemplary nucleic acid sequence, SEQ ID NO:17);
(2) VH #2
   QLQLQESGPGLVKPSETLSLTCTVSGGSISSSSYYWGWIRQPPGKGLE WIGSIYYSGSTYYNPSLKSRVTISVDTSKNQFSLKLSSVTAADTAVYYC ARLDPFDYWGRGTLVTVSS (SEQ ID NO:8; corresponding exemplary nucleic acid sequence, SEQ ID NO:18); or
a sequence having at least any of 80%, 85%, 90%, 95%, 97%, 98%, 99% or 99.5% sequence identify with the aforementioned sequences, provided the antigen binding domain binds to a citrullinated antigen as described herein.

The antigen binding region can comprise VL sequences selected from:
(1) VL #1
   SYVLTQPPSVSLAPGETATITCGGDDIENQNVNWYQQKSGQAPMLLIF FDTRRPSGIPERFSGSRSEDTANLTITRVEAGDDADYFCQVYDRKTDH QVFGPGTTVTVL (SEQ ID NO:9; corresponding exemplary nucleic acid sequence, SEQ ID NO:19);
(2) VL #2
   SYVLTQPPSVSVAPGKTARITCGGNNIGSKSVHWYQQKPGQAPVLVIY YDSDRPSGIPERFSGSNSGNTATLTISRVEAGDEADYYCQVWDSSSDH QVFGTGTKVTVL (SEQ ID NO:10; corresponding exemplary nucleic acid sequence, SEQ ID NO:20); or
a sequence having at least any of 80%, 85%, 90%, 95%, 97%, 98%, 99% or 99.5% sequence identify with the aforementioned sequences, provided the antigen binding domain binds to a citrullinated antigen as described herein.

In some embodiments, the antibody described above is the capture antibody or the detection antibody. In some embodiments, the antibody described above is the capture antibody. In other embodiments, the antibody described above is the detection antibody. In some embodiments, the antibody described above binds to different epitopes of the citrullinated polypeptide or citrullinated peptide from the F95 anti-peptidyl-citrulline antibody clone.

In some embodiments, the antibody described above is the capture antibody. In some embodiments, the capture antibody is immobilized to a sample plate. In some embodiments, the capture antibody is immobilized to a bead. In some embodiments, the detection antibody is an anti-peptidyl-citrulline antibody, for example the F95 antibody clone. In some embodiments, the capture antibody and the detection antibody bind to different epitopes of the citrullinated polypeptide or citrullinated peptide. In some embodiments, the capture antibody binds to one or more of: citrullinated vimentin, citrullinated filaggrin, citrullinated fibrinogen, citrullinated tenascin C, and/or citrullinated fragments thereof. In some embodiments, the capture antibody binds to two or more of: citrullinated vimentin, citrullinated filaggrin, citrullinated fibrinogen, citrullinated tenascin C, and/or citrullinated fragments thereof. In some embodiments, the capture antibody binds to three or more of: citrullinated vimentin, citrullinated filaggrin, citrullinated fibrinogen, citrullinated tenascin C, and/or citrullinated fragments thereof. In some embodiments, the capture antibody binds to four or more of: citrullinated vimentin, citrullinated filaggrin, citrullinated fibrinogen, citrullinated tenascin C, and/or citrullinated fragments thereof. In some embodiments, the capture antibody binds to each of: citrullinated vimentin, citrullinated filaggrin, citrullinated fibrinogen, citrullinated tenascin C, and citrullinated fragments thereof.

### C. Cell-Based Detection Assay

In one aspect is provided a method of detecting the presence of the amount of a citrullinated polypeptide or citrullinated peptide, for example a citrullinated protein or fragment thereof, in a biological sample obtained from an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with an autoimmune or inflammatory disease (e.g., rheumatoid arthritis or hidradenitis suppurativa) based upon the presence or amount of the citrullinated polypeptide or citrullinated peptide. In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurative.

In some embodiments, the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, synovial fluid, whole blood, serum, plasma, and any combination thereof. In some embodiments, the biological sample is serum. In some embodiments, the biological sample is synovial fluid. In some embodiments, the biological sample is serum and synovial fluid. In some embodiments, the serum and the synovial fluid are from the same individual. In some embodiments, the individual is a human. In some embodiments, the individual has rheumatoid arthritis.

In some embodiments, the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, synovial fluid, whole blood, serum, plasma, and any combination thereof. In some embodiments, the biological sample is serum. In some embodiments, the biological sample is pus. In some embodiments, the biological sample is sweat. In some embodiments, the biological sample is a lesional skin biopsy. In some embodiments, the biological sample is two or more of serum, pus, sweat, and/or lesional skin biopsy. In some embodiments, the two or more of serum, pus, sweat, and/or lesional skin biopsy are from the same individual. In some embodiments, the individual is a human. In some embodiments, the individual has hidradenitis suppurativa.

In some embodiments, the cell is a mammalian cell. The cell can be any mammalian cell known or commonly used in the art, for example an immortalized cell line. Examples of immortalized cell lines include, but are not limited to, any one of CHO cells, BHK cells, MDCK cells, C3H 10T½ cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, HEK293 cells, HEK293T cells, B-50 cells, 3T3 cells, NIH3T3 cells, HepG2 cells, Saos-2 cells, Huh7 cells, HeLa cells, W163 cells, PerC6 cells, NSO cells, 211 cells, 211 A cells, and Jurkat T cells. In some embodiments, the cell is an immune cell. In some embodiments, the cell is a T cell, such as any one of CD4+ T cell, CD8+ T cell, α/β T cell, γ/δ T cell, memory T cell, effector T cell, regulatory T cell, stemlike T cell, naïve T cell, etc. In some embodiments, the cell is a Jurkat T cell. In some embodiments, the Jurkat T cell is a reporter cell. In some embodiments, the Jurkat T cell produces a bioluminescent signal upon activation. In some embodiments, the Jurkat T cell produces luciferase protein upon activation. In some embodiments, the signal is derived from the activity of the luciferase protein.

In some embodiments, the Jurkat T cell comprises the CAR described in Section V below. In some embodiments, the CAR comprises an optional signal peptide, a CAR antigen binding domain that specifically binds to a citrullinated polypeptide or citrullinated peptide, an optional hinge domain, a transmembrane domain, one or more optional co-stimulatory domains, and an intracellular signaling domain.

In some embodiments, the antigen binding domain of the CAR binds to citrullinated polypeptides or citrullinated peptides. In some embodiments, the CAR binds to any one or more of citrullinated vimentin, citrullinated filaggrin, citrullinated fibrinogen, citrullinated tenascin C, and/or citrullinated fragments thereof. In some embodiments, the CAR binds to any two or more of citrullinated vimentin, citrullinated filaggrin, citrullinated fibrinogen, citrullinated tenascin C, and/or citrullinated fragments thereof. In some embodiments, the CAR binds to any three or more of citrullinated vimentin, citrullinated filaggrin, citrullinated fibrinogen, citrullinated tenascin C, and/or citrullinated fragments thereof. In some embodiments, the CAR binds to any four or more of citrullinated vimentin, citrullinated filaggrin, citrullinated fibrinogen, citrullinated tenascin C, and/or citrullinated fragments thereof. In some embodiments, the CAR binds to each of citrullinated vimentin, citrullinated filaggrin, citrullinated fibrinogen, citrullinated tenascin C, and/or citrullinated fragments thereof.

In some embodiments, the CAR antigen binding domain comprises an antibody fragment selected from the group consisting of an Fv, scFv, camelid nanobody, heavy chain only antibody, or aptamer. In some embodiments, the antigen binding domain of the CAR comprises the same VH and VL as the capture antibody or the detection antibody described in Section II above. In some embodiments, the antigen binding domain of the CAR comprises an scFV comprising a VH and a VL joined by a linker selected from the group consisting of SEQ ID NOs:11-13. In some embodiments, the CAR antigen binding domain comprises the amino acid sequence of any one of SEQ ID NOs:14-16. In some embodiments, binding of the target antigen to the CAR's antigen binding domain induces CAR Jurkat T cell activation. In some embodiments, the induction of CAR Jurkat T cell activation produces a reporter molecule. In some embodiments, the reporter molecule is any suitable molecule, including but not limited to, a chemiluminescent enzyme (e.g., luciferase protein and alkaline phosphatase enzyme) and fluorescent protein (e.g., green fluorescent protein). For example, luciferase protein binds to a substrate molecule (e.g., luciferin) to get converted into a detectable product (e.g., light). In some embodiments, the light produced by luciferase protein is detected using any method known in the art, for example a luminometer or photographic film. In some embodiments, detection of the presence or amount of luciferase is a readout for the detection of the presence or amount of a citrullinated polypeptide or a citrullinated peptide.

In some embodiments, the intracellular signaling domain of the CAR is derived from CD3-zeta or an Fc receptor. In some embodiments, the CAR comprises at least one co-stimulatory domain derived from the group consisting of: CD3-zeta, FceR1g, Fcg, CD28, 4-1BB, OX40, CD40L, MyD88, CD40, ICOS, RANK/TRANCE-R, CTLA-4, CTLA-4/CD-28 hybrid, DAP10, CD27, and 2B4. In some embodiments, the CAR further comprises a hinge domain as described in Section V.

### III. Methods of Diagnosing Autoimmune or Inflammatory Diseases

Provided herein are methods of diagnosing an autoimmune or inflammatory disease (e.g., rheumatoid arthritis or hidradenitis suppurativa) in an individual. For example, detecting citrullinated peptides and/or citrullinated polypeptides can be used to determine whether an individual has an autoimmune or inflammatory disease (e.g., rheumatoid arthritis or hidradenitis suppurativa), wherein the presence or an increased amount of citrullinated peptides and/or citrullinated polypeptides indicates that the individual has the autoimmune or inflammatory disease (e.g., rheumatoid arthritis or hidradenitis suppurativa). In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurative. In some embodiments, the autoimmune disease or inflammatory disease is rheumatoid arthritis. In some embodiments, the autoimmune or inflammatory disease is hidradenitis suppurativa.

In one aspect is provided a method of diagnosing an autoimmune or inflammatory disease (e.g., rheumatoid arthritis or hidradenitis suppurativa) in an individual comprising a) incubating a biological sample obtained from the individual with a capture antibody that binds to a citrullinated polypeptide or citrullinated peptide to form a complex comprising the capture antibody and the citrullinated polypeptide or citrullinated peptide; b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with an autoimmune or inflammatory disease (e.g., rheumatoid arthritis or hidradenitis suppurativa) based upon the presence or amount of the citrullinated polypeptide or citrullinated peptide.

In a second aspect is provided a method of diagnosing an autoimmune or inflammatory disease (e.g., rheumatoid arthritis or hidradenitis suppurativa) in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with an autoimmune or inflammatory disease (e.g., rheumatoid arthritis or hidradenitis suppurativa) based upon the presence or amount of the citrullinated polypeptide or citrullinated peptide.

In one aspect is provided a method of diagnosing rheumatoid arthritis in an individual comprising a) incubating a biological sample obtained from the individual with a capture antibody that binds to a citrullinated polypeptide or citrullinated peptide to form a complex comprising the capture antibody and the citrullinated polypeptide or citrullinated peptide; b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with rheumatoid arthritis based upon the presence or amount of the citrullinated polypeptide or citrullinated peptide. In a second aspect is provided a method of diagnosing rheumatoid in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with rheumatoid arthritis based upon the presence or amount of the citrullinated polypeptide or citrullinated peptide.

In one aspect is provided a method of diagnosing hidradenitis suppurativa in an individual comprising a) incubating a biological sample obtained from the individual with a capture antibody that binds to a citrullinated polypeptide or citrullinated peptide to form a complex comprising the capture antibody and the citrullinated polypeptide or citrullinated peptide; b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with hidradenitis suppurativa based upon the presence or amount of the citrullinated polypeptide or citrullinated peptide. In a second aspect is provided a method of diagnosing hidradenitis suppurativa in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and b) detecting the signal emitted by the CAR to determine the presence or amount of the citrullinated polypeptide or citrullinated peptide, wherein the individual is diagnosed with hidradenitis suppurativa based upon the presence or amount of the citrullinated polypeptide or citrullinated peptide.

In some embodiments, the methods of diagnosing further comprise comparing the amount of the citrullinated polypeptide or citrullinated peptide in the biological sample to a reference level or reference sample. In some embodiments, the reference sample is a biological sample obtained from an individual not having an autoimmune or inflammatory disease (e.g., rheumatoid arthritis or hidradenitis suppurativa). In some cases, the reference sample may be used to identify a threshold value of the amount of the citrullinated polypeptide or citrullinated peptide over which an individual is diagnosed with, selected for treatment of, or otherwise indicated as likely to suffer from an autoimmune or inflammatory disease (e.g., rheumatoid arthritis or hidradenitis suppurativa). In some cases, a value that falls above this threshold indicates the presence of citrullinated polypeptides or citrullinated peptides. In some embodiments, the presence of citrullinated polypeptides or citrullinated peptides or amount of citrullinated polypeptides or citrullinated peptides that is above a reference level or threshold derived therefrom is indicative of an autoimmune or inflammatory disease (e.g., rheumatoid arthritis or hidradenitis suppurativa). In some embodiments, a diagnosis of an autoimmune or inflammatory disease (e.g., rheumatoid arthritis or hidradenitis suppurativa) is made upon detection of the presence or amount of citrullinated polypeptide or citrullinated peptide in an individual.

Diagnosing an individual with an autoimmune or inflammatory disease (e.g., rheumatoid arthritis or hidradenitis suppurativa) can also include testing for additional factors known in the art as being indicative of, diagnostic of, or a hallmark for the autoimmune or inflammatory disease (e.g., rheumatoid arthritis or hidradenitis suppurative). Such factors can be identified by imaging (for example, radiological imaging, MRI, CT scan, X-ray, ultrasound), mobility or pain scores, identification of markers of inflammation (for example, serum or synovial fluid levels of inflammatory molecules, such as any one or more of C reactive protein (CRP), IL-1β, IL-6, IL-8, IL-17, IL-23, IL-18, IL-12p70, IP-10, MCP-1, MIP-1a, IFN-γ, and TNF-α), detection of autoantibodies (for example, ACPA or rheumatoid factor (RF)), etc. In some embodiments, the methods of diagnosing an autoimmune or inflammatory disease (e.g., rheumatoid arthritis or hidradenitis suppurativa) in an individual further comprise: a) detecting the level of one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in a biological sample; b) comparing the level of the one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α to a reference level; and c) selecting the individual as having the autoimmune or inflammatory disease (e.g., rheumatoid arthritis or hidradenitis suppurativa) when the level of the one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in the biological sample is above the reference level.

In some embodiments, there is provided a method of diagnosing rheumatoid arthritis in an individual further comprise: a) detecting the level of one or more proteins selected from the group consisting of CRP, IL-1β, IL-6, IL-18, IL-12p70, IP-10, MCP-1, MIP-1a, and TNF-α in a biological sample; b) comparing the level of the one or more proteins to a reference level; and c) selecting the individual as having rheumatoid arthritis when the level of the one or more proteins in the biological sample is above the reference level.

In some embodiments, there is provided a method of diagnosing hidradenitis suppurativa in an individual further comprise: a) detecting the level of one or more proteins selected from the group consisting of IL-1β, IL-6, IL-8, IL-17, IL-23, IP-10, IFN-γ, and TNF-α in a biological sample; b) comparing the level of the one or more proteins to a reference level; and c) selecting the individual as having hidradenitis suppurativa when the level of the one or more proteins in the biological sample is above the reference level.

An individual suspected of having an autoimmune or inflammatory disease (e.g., rheumatoid arthritis or hidradenitis suppurativa) can be tested for diagnosis at any point after the onset of symptoms indicative of the autoimmune or inflammatory disease (e.g., rheumatoid arthritis or hidradenitis suppurativa). For example, the methods of diagnosing described herein can be utilized within about any one or more of 1 day, 2 days, 3 days, 4 days, 5 days, 6 days, 7 days, 8 days, 9 days, 10 days, 11 days, 12 days, 13 days, 14 days, 15 days, 1 week, 2 weeks, 3 weeks, 4 weeks, 5 weeks, 6 weeks, 7 weeks, 8 weeks, 8 weeks, 10 weeks, 11 weeks, 12 weeks, 13 weeks, 14 weeks, 15 weeks, 16 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 1 year, 2 years, 3 years, 4 years, 5 years, 6 years, 7 years, 8 years, 9 years, 10 years or more after the onset of symptoms indicative of the autoimmune or inflammatory disease (e.g., rheumatoid arthritis or hidradenitis suppurativa).

In some embodiments, the methods of diagnosing can further comprise selecting an individual for treatment and/or treating an individual with an autoimmune or inflammatory disease (e.g., rheumatoid arthritis or hidradenitis suppurativa) as described below.

### IV. Methods of Selecting an Individual with Autoimmune or Inflammatory Disease for Treatment

Provided herein are methods of selecting an individual having an autoimmune or inflammatory disease (e.g., rheumatoid arthritis or hidradenitis suppurativa) for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level. The detecting the presence or amount of the citrullinated polypeptide or citrullinated peptide in a biological sample obtained from the individual can include any of the methods of detecting described in the present application. In some aspects, one advantage of the present method is that the assays used herein may use an antibody or CAR that comprises the same antigen binding domain as a CAR used in a cell therapy to treat the autoimmune disease or inflammatory disease, e.g., rheumatoid arthritis (RA). This allows confirmation that the individual expresses the target epitope of the cell therapy prior to administration of the cell therapy. In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurative.

In some embodiments, the method further comprises comparing the amount of the citrullinated polypeptide or citrullinated peptide in the biological sample to a reference level or reference sample. In some embodiments, the reference sample is a biological sample obtained from an individual not having an autoimmune or inflammatory disease (e.g., rheumatoid arthritis or hidradenitis suppurativa). In some cases, the reference sample may be used to identify a threshold value of the amount of the citrullinated polypeptide or citrullinated peptide over which an individual is diagnosed with, selected for treatment of, or otherwise indicated as likely to suffer from an autoimmune or inflammatory disease (e.g., rheumatoid arthritis or hidradenitis suppurativa). In some cases, a value that falls above this threshold indicates the presence of citrullinated polypeptides or citrullinated peptides.

In some embodiments, there is provided a method of selecting an individual having rheumatoid arthritis for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level. In some embodiments, the method further comprises comparing the amount of the citrullinated polypeptide or citrullinated peptide in the biological sample to a reference level or reference sample. In some embodiments, the reference sample is a biological sample obtained from an individual not having rheumatoid arthritis. In some cases, the reference sample may be used to identify a threshold value of the amount of the citrullinated polypeptide or citrullinated peptide over which an individual is diagnosed with, selected for treatment of, or otherwise indicated as likely to suffer from rheumatoid arthritis. In some cases, a value that falls above this threshold indicates the presence of citrullinated polypeptides or citrullinated peptides.

In some embodiments, there is provided a method of selecting an individual having hidradenitis suppurativa for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level. In some embodiments, the method further comprises comparing the amount of the citrullinated polypeptide or citrullinated peptide in the biological sample to a reference level or reference sample. In some embodiments, the reference sample is a biological sample obtained from an individual not having hidradenitis suppurativa. In some cases, the reference sample may be used to identify a threshold value of the amount of the citrullinated polypeptide or citrullinated peptide over which an individual is diagnosed with, selected for treatment of, or otherwise indicated as likely to suffer from hidradenitis suppurativa. In some cases, a value that falls above this threshold indicates the presence of citrullinated polypeptides or citrullinated peptides.

Selecting an individual for treatment can further include testing for additional factors known in the art as being indicative of, diagnostic of, or a hallmark for the autoimmune or inflammatory disease (e.g., rheumatoid arthritis or hidradenitis suppurativa). Such factors can be identified by imaging (for example, radiological imaging, MRI, CT scan, X-ray, ultrasound), mobility or pain scores, identification of markers of inflammation (for example, serum or synovial fluid levels of inflammatory molecules), detection of autoantibodies (for example, ACPA or rheumatoid factor (RF)), etc. For example, possible markers of rheumatoid arthritis can include, but are not limited to, elevation in any one or more cytokines selected from the group consisting of C reactive protein (CRP), IL-1β, IL-6, IL-18, IL-12p70, IP-10, MCP-1, MIP-1a, and TNF-α. As another example, possible markers for hidradenitis suppurativa can include, but are not limited to, elevation in any one or more cytokines selected from the group consisting of IL-1β, IL-6, IL-8, IL-17, IL-23, IP-10, IFN-γ, and TNF-α. In some embodiments, the methods of selecting an individual having an autoimmune or inflammatory disease (e.g., rheumatoid arthritis or hidradenitis suppurativa) further comprise: a) detecting the level of one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in a biological sample; b) comparing the level of the one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α to a reference level; and c) selecting the individual as having the autoimmune or inflammatory disease (e.g., rheumatoid arthritis or hidradenitis suppurativa) when the level of the one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in the biological sample is above the reference level.

In some embodiments, there is provided a method of selecting an individual having rheumatoid arthritis for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level, wherein the method further comprises: i) detecting the level of one or more proteins selected from the group consisting of CRP, IL-1β, IL-6, IL-18, IL-12p70, IP-10, MCP-1, MIP-1a, and TNF-α in a biological sample; b) comparing the level of the one or more proteins to a reference level; and c) selecting the individual as having rheumatoid arthritis when the level of the one or more proteins in the biological sample is above the reference level.

In some embodiments, there is provided a method of selecting an individual having hidradenitis suppurativa for treatment with a T cell therapy comprising a T cell comprising a chimeric antigen receptor (CAR) that binds to a citrullinated polypeptide or citrullinated peptide comprising a) detecting the presence or amount of the citrullinated polypeptide or citrullinated peptide in a biological sample obtained from the individual; and b) selecting the individual for treatment with the T cell therapy if the biological sample comprises citrullinated polypeptides or citrullinated peptides or an amount of citrullinated polypeptides or citrullinated peptides that is above a threshold or a reference level, wherein the method further comprises: i) detecting the level of one or more proteins selected from the group consisting of IL-1β, IL-6, IL-8, IL-17, IL-23, IP-10, IFN-γ, and TNF-α in a biological sample; b) comparing the level of the one or more proteins to a reference level; and c) selecting the individual as having hidradenitis suppurativa when the level of the one or more proteins in the biological sample is above the reference level.

### V. Method of Treating Autoimmune or Inflammatory Diseases

Provided herein are methods of treating an autoimmune or inflammatory disease (e.g., rheumatoid arthritis or hidradenitis suppurativa) in an individual. In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurative.

In one aspect is provided a method of treating an autoimmune or inflammatory disease (e.g., rheumatoid arthritis or hidradenitis suppurativa) in an individual comprising i) detecting a citrullinated polypeptide or citrullinated peptide in a biological sample obtained from an individual comprising a) incubating the biological sample with a capture antibody that binds to a citrullinated polypeptide or citrullinated peptide to form a complex comprising the capture antibody and the citrullinated polypeptide or citrullinated peptide, b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody, and c) detecting the complex comprising the capture antibody, the citrullinated polypeptide or the citrullinated peptide, and the detection antibody; and ii) administering to the individual an effective amount of a composition comprising a T cell comprising a chimeric antigen receptor (CAR) comprising an antigen binding domain that specifically binds to a citrullinated polypeptide or citrullinated peptide, if the biological sample comprises citrullinated polypeptides or citrullinated peptides or an amount of citrullinated polypeptides or citrullinated peptides that is above a threshold or a reference level. In some embodiments, the autoimmune or inflammatory disease is rheumatoid arthritis. In some embodiments, the autoimmune or inflammatory disease is hidradenitis suppurativa.

In another aspect is provided a method of treating an autoimmune or inflammatory disease (e.g., rheumatoid arthritis or hidradenitis suppurativa) in an individual comprising a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; b) detecting the signal emitted by the CAR to determine the presence or amount of the citrullinated polypeptide or citrullinated peptide; and c) administering to the individual an effective amount of a composition comprising a T cell comprising a chimeric antigen receptor (CAR) comprising an antigen binding domain that specifically binds to a citrullinated polypeptide or citrullinated peptide if the biological sample comprises citrullinated polypeptides or citrullinated peptides or an amount of citrullinated polypeptides or citrullinated peptides that is above a threshold or a reference level. In some embodiments, the autoimmune or inflammatory disease is rheumatoid arthritis. In some embodiments, the autoimmune or inflammatory disease is hidradenitis suppurativa.

### A. Chimeric Antigen Receptors

In some embodiments, the individual is administered an effective amount of a composition comprising a T cell comprising a chimeric antigen receptor (CAR) comprising an antigen binding domain that specifically binds to a citrullinated polypeptide and/or citrullinated peptide. “Chimeric antigen receptors” or “CARs” are engineered molecules comprising an optional signal peptide, an antigen binding domain, an optional hinge domain or hinge region, a transmembrane domain, an intracellular signaling domain and an optional co-stimulatory domain. CARs are based on the structure of T cell receptors, which are expressed on T cells, and which are involved in the cell-mediated immune response.

So-called “first-generation” CARs had an antigen domain and a CD3 signal transduction domain. So-called “second generation” CARs further included a co-stimulatory domain, such as a CD28 or 4-1BB domain. So-called “third generation” CARs comprise multiple co-stimulatory domains. So-called “fourth generation” CARs, also referred to as “TRUCKS”, are engineered to release a transgenic cytokine upon CAR signaling.

Chimeric antigen receptors (“CARs”) include the following elements: (1) an optional signal peptide, (2) an antigen binding domain, (3) an optional hinge domain or hinge region; (4) a transmembrane region; and (5) an intracellular domain comprising a signal transduction domain. Optionally, the CAR can include any of: a CD3ζ signal transduction domain, an Fc receptor signal transduction domain, a co-stimulatory (signal transduction) domain. That is, these optional elements can be included in addition to or instead of other optional elements. The CAR antigen binding domain is heterologous to at least one of the other domains. That is, the CAR antigen binding domain does not naturally occur on a T cell receptor.

The “antigen binding domain” provides binding specificity to the CAR. The “signal peptide” guides the polypeptide through the cell membrane. The CAR antigen binding domain can bind to a domain of an antibody that binds to the target antigen for a so-called “Universal CAR”. The “hinge region” or “hinge domain” (terms used interchangeably) is a flexible connector region, e.g., a natural or synthetic polypeptide, or any other type of molecule, providing structural flexibility and spacing to flanking polypeptide regions. The “transmembrane domain” is a membrane-spanning protein domain, typically hydrophobic. The “signal transduction domain” or “signaling domain” transmits a signal through a signal transduction pathway into the cell upon binding. Such signaling activates an activity of the cell. “Co-stimulatory domains” are accessory signaling domains that further transmit signals.

### 1. Signal Peptide

A signal peptide can be any peptide having the function of allowing a polypeptide to traverse a cell membrane. The signal peptide can be derived from CD4, CD8, CD28, TLR or immunoglobulin family of receptors.

An exemplary signal peptide is:

(SEQ ID NO: 39)
MALPVTALLLPLALLLHAAR.

### 2. CAR Antigen Binding Domain

### a) Structure

The CAR antigen binding domain can include any polypeptide comprising an antigen binding function, e.g., an antibody as defined herein. In one embodiment, the CAR antigen binding domain can comprise a single chain antibody (scFV). The scFv can be connected to the transmembrane domain via a hinge domain whose length, flexibility and origin provides variability in the CAR's design, and can, along with the transmembrane domain, contribute to the interaction with antigen, building of the immunologic synapse and impact the CAR's association with additional proteins needed to impart a robust activation signal.

### b) Targets

Chimeric antigen receptors disclosed herein comprise an antigen binding domain that binds to citrullinated antigens, e.g., those found in the synovium of subjects with rheumatoid arthritis, the skin lesions of subjects with hidradenitis suppurativa, the myelin sheath or spinal cord white matter of subjects with multiple sclerosis, etc. In particular, the CAR antigen binding domain can bind to one or more of (i) citrullinated vimentin, (ii) citrullinated filaggrin, (iii) citrullinated fibrinogen and (iv) citrullinated peptides thereof. In some embodiments, the CAR antigen binding domain can bind to a citrullinated peptide fragment of (i)-(iii), wherein the peptide fragment is at least of 10 amino acids in length, e.g., of at least about 12 amino acids, at least about 14 amino acids, at least about 16 amino acids in length, or more. In some embodiments, the CAR antigen binding domain further binds to tenascin C. In some embodiments, the CAR antigen binding domain can bind to two or more of (i) citrullinated vimentin, (ii) citrullinated filaggrin, (iii) citrullinated fibrinogen, (iv) tenascin C, or (v) citrullinated peptide fragments thereof. In some embodiments, the target antigen is one or more citrullinated peptides selected from the following sequences:

(SEQ ID NO: 27)
ST(Cit)SVSSSSY(Cit)(Cit)MFGG
(SEQ ID NO: 28)
VYAT(Cit)SSAV(Cit)L(Cit)SSV
(SEQ ID NO: 29)
(Cit)PAPPPISGGGY(Cit)A(Cit)
(SEQ ID NO: 30)
SHQEST(Cit)GRSRGRSGRSGS

The CAR antigen binding domain can comprise sequences from an antibody VH and VL domain. This includes particular CDR sets from VH and VL domains. In some embodiments, the CAR antigen binding domain comprises the CDRs from the VH domains of SEQ ID NO:7 or SEQ ID NO:8. In some embodiments, the CAR antigen binding domain comprises the CDRs from the VL domains of SEQ ID NO:9 or SEQ ID NO:10. In some embodiments, the CAR antigen binding domain comprises the CDRs from VH and VL domains of SEQ ID NO:7 and SEQ ID NO:9, respectively. In some embodiments, the CAR antigen binding domain comprises the CDRs from VH and VL domains of SEQ ID NO:7 and SEQ ID NO: 10, respectively. In some embodiments, the CAR antigen binding domain comprises the CDRs from VH and VL domains of SEQ ID NO:8 and SEQ ID NO:9, respectively. In some embodiments, the CAR antigen binding domain comprises the CDRs from VH and VL domains of SEQ ID NO:8 and SEQ ID NO: 10, respectively.

In some embodiments, the CAR antigen binding domain comprises the complementarity-determining regions (CDRs) from VH and VL domains of SEQ ID NO:7 and SEQ ID NO:10. In some embodiments, the VH domain of the target-binding domain comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and the VL domain of the target-binding domain comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6.

| | SEQ | | | |
|---|---|---|---|---|
| Region | ID | Sequence Fragment | Residues | Length |
| HFR1 | NO: 31 | HLHLQESGPGLVKPSETLSLTCTVSGGSIN | 1-30 | 30 |
| CDR- | NO: 1 | DTTYYWG | 31-37 | 7 |
| H1 | | | | |
| HFR2 | NO: 32 | WIRQPPGKGLEWIG | 38-51 | 14 |
| CDR- | NO: 2 | SIYYRGNTHYNSSLRS | 52-67 | 16 |
| H2 | | | | |
| HFR3 | NO: 33 | RVTMSVDTSKNRFSLKVTSVTAADTAVYYCA | 68-99 | 32 |
| | | R | | |
| CDR- | NO: 3 | LDPFDY | 100-105 | 6 |
| H3 | | | | |
| HER4 | NO: 34 | WGRGTLVTVSS | 106-116 | 11 |
| LFR1 | NO: 35 | SYVLTQPPSVSVAPGKTARITC | 1-22 | 22 |
| CDR- | NO: 4 | GGNNIGSKSVH | 23-33 | 11 |
| L1 | | | | |
| LFR2 | NO: 36 | WYQQKPGQAPVLVIY | 34-48 | 15 |
| CDR- | NO: 5 | YDSDRPS | 49-55 | 7 |
| L2 | | | | |
| LFR3 | NO: 37 | GIPERFSGSNSGNTATLTISRVEAGDEADYYC | 56-87 | 32 |
| CDR- | NO: 6 | QVWDSSSDHQV | 88-98 | 11 |
| L3 | | | | |
| LFR4 | NO: 38 | FGTGTKVTV | 99-108 | 11 |

In certain embodiments, the CAR antigen binding domain comprises VH sequences selected from:
(1) VH #1
   HLHLQESGPGLVKPSETLSLTCTVSGGSINDTTYYWGWIRQPPGKGLE WIGSIYYRGNTHYNSSLRSRVTMSVDTSKNRFSLKVTSVTAADTAVY YCARLDPFDYWGRGTLVTVSS (SEQ ID NO:7; corresponding exemplary nucleic acid sequence, SEQ ID NO:17);
(2) VH #2
   QLQLQESGPGLVKPSETLSLTCTVSGGSISSSSYYWGWIRQPPGKGLE WIGSIYYSGSTYYNPSLKSRVTISVDTSKNQFSLKLSSVTAADTAVYYC ARLDPFDYWGRGTLVTVSS (SEQ ID NO:8; corresponding exemplary nucleic acid sequence, SEQ ID NO:18); or
a sequence having at least any of 80%, 85%, 90%, 95%, 97%, 98%, 99% or 99.5% sequence identify with the aforementioned sequences, provided the CAR antigen binding domain binds to a citrullinated antigen as described herein.

The CAR antigen binding region can comprise VL sequences selected from:
(1) VL #1
   SYVLTQPPSVSLAPGETATITCGGDDIENQNVNWYQQKSGQAPMLLIF FDTRRPSGIPERFSGSRSEDTANLTITRVEAGDDADYFCQVYDRKTDH QVFGPGTTVTVL (SEQ ID NO:9; corresponding exemplary nucleic acid sequence, SEQ ID NO:19);
(2) VL #2
   SYVLTQPPSVSVAPGKTARITCGGNNIGSKSVHWYQQKPGQAPVLVIY YDSDRPSGIPERFSGSNSGNTATLTISRVEAGDEADYYCQVWDSSSDH QVFGTGTKVTVL (SEQ ID NO:10; corresponding exemplary nucleic acid sequence, SEQ ID NO:20); or
a sequence having at least any of 80%, 85%, 90%, 95%, 97%, 98%, 99% or 99.5% sequence identify with the aforementioned sequences, provided the CAR antigen binding domain binds to a citrullinated antigen as described herein.

In another embodiment, the CAR antigen binding region comprises an scFV comprising an amino acid sequence selected from:
VHVL—GGGSx3 Linker

(SEQ ID NO: 14)
HLHLQESGPGLVKPSETLSLTCTVSGGSINDTTYYWGWIRQPPGKGLE
WIGSIYYRGNTHYNSSLRSRVTMSVDTSKNRFSLKVTSVTAADTAVY
YCARLDPFDYWGRGTLVTVSSGGGGSGGGGSGGGGSSYVLTQPPSV
SVAPGKTARITCGGNNIGSKSVHWYQQKPGQAPVLVIYYDSDRPSGIP
ERFSGSNSGNTATLTISRVEAGDEADYYCQVWDSSSDHQVFGTGTKV
TVLR

(GGGGSx3 linker underlined; corresponding exemplary nucleic acid sequence, SEQ ID NO:24);
VHVL—Whitlow 218 Linker

(SEQ ID NO: 15)
HLHLQESGPGLVKPSETLSLTCTVSGGSINDTTYYWGWIRQPPGKGLE
WIGSIYYRGNTHYNSSLRSRVTMSVDTSKNRFSLKVTSVTAADTAVY
YCARLDPFDYWGRGTLVTVSSGSTSGSGKPGSGEGSTKGSYVLTQPP
SVSVAPGKTARITCGGNNIGSKSVHWYQQKPGQAPVLVIYYDSDRPSG
IPERFSGSNSGNTATLTISRVEAGDEADYYCOVWDSSSDHQVFGTGTK
VTVLR

(Whitlow 218 Linker underlined; corresponding exemplary nucleic acid sequence, SEQ ID NO:25);
VHVL—AB pur Linker

(SEQ ID NO: 16)
HLHLQESGPGLVKPSETLSLTCTVSGGSINDTTYYWGWIRQPPGKGLE
WIGSIYYRGNTHYNSSLRSRVTMSVDTSKNRFSLKVTSVTAADTAVY
YCARLDPFDYWGRGTLVTVSSASSGGSTSGSGKPGSGEGSSGSARSY
VLTQPPSVSVAPGKTARITCGGNNIGSKSVHWYQQKPGQAPVLVIYYD
SDRPSGIPERFSGSNSGNTATLTISRVEAGDEADYYCQVWDSSSDHQV
FGTGTKVTVLR

(AB pur Linker underlined; corresponding exemplary nucleic acid sequence, SEQ ID NO:26); or
a sequence having at least any of 80%, 85%, 90%, 95%, 97%, 98%, 99% or 99.5% sequence identify with the aforementioned sequences, provided the CAR antigen binding domain binds to a citrullinated antigen as described herein.

Accordingly, exemplary linkers are:
GGGGSGGGGSGGGGS (SEQ ID NO:11) GGGGSx3 Linker (corresponding exemplary nucleic acid sequence, SEQ ID NO:21);
GSTSGSGKPGSGEGSTKG (SEQ ID NO:12) Whitlow 218 Linker (corresponding exemplary nucleic acid sequence, SEQ ID NO:22); or
ASSGGSTSGSGKPGSGEGSSGSAR (SEQ ID NO:13) AB pur Linker (corresponding exemplary nucleic acid sequence, SEQ ID NO:23).

Optionally, any of the foregoing sequences can include the CDR sets described above.

### 3. Hinge Domain or Region

In some embodiments, the hinge domain (i.e., hinge region) of the disclosed CARs can be selected from the CD8, CD4, or CD28 extracellular domain, the Fc region of an IgG1 antibody, or the extracellular domain of any of the TLR receptors as is known to one of skill in the art and can be found in the GenBank database. In some embodiments, the hinge domain or region is any suitable hinge domain or region known in the art.

For example, the hinge domain or region can comprise the sequence:

(CD28)
(SEQ ID NO: 40)
IEVMYPPPYLDNEKSNGTIIHVKGKHLCPSPLFPGPSKP;
(CD8)
(SEQ ID NO: 41)
TTTPAPRPPTPAPTIASQPLSLRPEACRPAAGGAVHTRGLDFACDIYIW
APLAGTCGVLLLSLVITLYC;
or

a sequence having at least any of 80%, 85%, 90%, 95%, 97%, 98%, 99% or 99.5% sequence identify with the aforementioned hinge domain or region sequences.

### 4. Transmembrane Domain

The transmembrane domain can comprise a transmembrane domain of an immunoglobulin family receptor, such as CD8. The intracellular domain can be selected from any membrane-spanning molecule on a T cell. For example, the transmembrane (TM) domain of the disclosed CAR can comprise the TM domain selected from the group consisting of CD2, CD3, CD16, CD32, CD64, CD28, CD247, 4-1BBL, CD4, and CD8.

For example, the transmembrane domain can comprise a sequence selected from:

(SEQ ID NO: 42)
FWVLVVVGGVLACYSLLVTVAFIIFWV;
(SEQ ID NO: 43)
IYIWAPLAGTCGVLLLSLVITLYC;
or

a sequence having at least any of 80%, 85%, 90%, 95%, 97%, 98%, 99% or 99.5% sequence identify with the aforementioned transmembrane domain sequences.

### 5. Signal Transduction Domain

### a) CD3ζ Signal Transduction Domain

In some embodiments, the signal transduction domain comprises a CD3ζ signaling domain. The CD3ζ signaling domain of the disclosed CAR molecule can comprise a CD3ζ amino acid sequence, e.g., a signal transduction domain of CD3 zeta.

For example, the CD3ζ signal transduction domain can have a sequence selected from:

(SEQ ID NO: 44)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR;
or

a sequence having at least any of 80%, 85%, 90%, 95%, 97%, 98%, 99% or 99.5% sequence identify with the aforementioned CD3 zeta signal transduction domain sequence.

For example, the CD3 zeta signal transduction domain can include amino acids 21-163, 31-142, 68-89 and/or 138-158 of the sequence shown in SEQ ID NO:44, or functional variants thereof (e.g., with 1, 2, 3, 4, 5, 6, 7, 8, 9, 10, or 10-20 amino acid substitutions, deletions, or additions).

### b) Fc Receptor Signal Transduction Domain

In some embodiments, the signal transduction domain comprises an Fc signaling domain. The Fc signaling domain can be any one of the Fc-alpha, Fc-gamma, Fc-epsilon, Fc-mu, and Fc-delta receptors. For example, the Fc receptor signaling domain can comprise amino acids involved in interaction with Src (e.g., Fgr, Fyn, Hck, Lyn, Yes, and Src) and ZAP-70 family kinases, e.g., one or more ITAM domains (see, e.g., Sanchez-Mejorada et al. (1998) J. Leukocyte Biol. 63:531; Garcia-Garcia et al. (2002) J. Leukocyte Biol. 72:1092). In some embodiments, the Fc receptor signal transduction domain includes at least one ITAM domain, e.g., from any one of the Fc-alpha, Fc-gamma, Fc-epsilon, Fc-mu, and Fc-delta receptors, or substantially identical thereto.

Sequences also can be found as follows:

| | |
|---|---|
| CD3ζ | NP_000725.1 |
| Fc gamma receptor | NP_000560.5 |
| family (CD16) | NP_001231682.1 |
| Fc gamma receptor | AAH20823.1 |
| family (CD32) | AAH19931.1 |
| | AAI48274.1 |
| | AAI37398.1 |
| Fc gamma receptor | AAI60240.1 |
| family (CD64) | AAH32634.1 |
| | AAI56865.1 |

### 6. Co-Stimulatory Domain

The CARs of this disclosure can include one or more co-stimulatory domains in addition to a signal transduction domain of CD3ζ or an Fc receptor. Co-stimulatory domains can be derived from, for example, CD28, 4-1BB, CD2, CD27, CD30, OX40, CD40, PD-1, PD-L1, PD-L2, ICOS, LFA-1, CD7, LIGHT, NKG2C, B7-H3, CD83L, B7-1 (CD80), B7-2 (CD86), B7-H3, B7-H4, and others. The CAR constructs can contain two or more co-stimulatory signaling domains (e.g., CD28 and 4-1BB).

The co-stimulatory domain or domains can be positioned between the signal transduction domain and the transmembrane region.

In certain embodiments, a CD28 co-stimulatory domain can comprise a sequence selected from:

(SEQ ID NO: 45)
RSKRSRLLHSDYMNMTPRRPGPTRKHYQPYAPPRDFAAYRS;
or

a sequence having at least any of 80%, 85%, 90%, 95%, 97%, 98%, 99% or 99.5% sequence identify with the aforementioned sequence.

In certain embodiments, a 4-1BB co-stimulatory domain can comprise a sequence selected from:

(SEQ ID NO: 46)
KRGRKKLLYIFKQPFMRPVQTTQEEDGCSCRFPEEEEGGCEL;
or

a sequence having at least any of 80%, 85%, 90%, 95%, 97%, 98%, 99% or 99.5% sequence identify with the aforementioned sequence.

### B. Cells

This disclosure provides cells (e.g., recombinant cells) comprising any of the CARs described herein.

The cell can include, for example, any immune cell including but not limited to a T-cell, a CD4 T-cell, a Treg cell, a CD8 T-cell, a T helper cell, a granulocyte (e.g., neutrophils, basophils, eosinophils), a megakaryocyte, a monocyte, a macrophage, a dendritic cell, a T memory stem cell, as well as cells expressing MHC class I or class II as is known to one of skill in the art. In some embodiments, the cell comprising the disclosed CAR can be a myeloid progenitor cell selected from the group consisting of a common myeloid progenitor, a granulocyte macrophage progenitor, a megakaryocyte erythrocyte progenitor, a granulocyte progenitor and a monocyte progenitor as is known to one of skill in the art. In some embodiments, the myeloid cell is an autologous or allogeneic cell.

In some embodiments, the cells expressing the CARs of this disclosure are Treg cells. “Regulatory T cells,” or “T_{reg} cells,” are cells belonging to a specialized subpopulation of T cells that act to suppress immune response, thereby maintaining homeostasis and self-tolerance. T_{reg}s are able to inhibit T cell proliferation and cytokine production and play a critical role in preventing autoimmunity. T_{reg}s are characterized by expression of FoxP3. Surface markers for T_{reg}s include CD4, CD25high (high molecular density) and CD127low (low molecular density). Mouse and human Tregs express GITR/AITR, and CTLA-4. Human CD4+FoxP3+ Treg cells can be divided into three sub-populations: (1) CD45RA+CD25+FoxP3low resting Treg cells, (2) CD45RO+CD25highFoxP3high activated Treg cells, and (3) proinflammatory cytokine-producing CD45RO+CD25+FoxP3low nonsuppressive effector T cells (Teffs).

The cells to be administered to the individual as disclosed herein can be cells taken from the same individual into whom the CAR-expressing cells are to be administered. In this way, issues of an allogeneic immune response can be mitigated using an autologous approach.

In some instances, the cells to be administered to the individual as disclosed herein can be cells taken from a different individual than the individual into whom the CAR-expressing cells are to be administered. In this way, issues of acquiring immune cells can be mitigated using an “off the shelf” allogeneic approach.

Cells can be expanded ex vivo before administration to a subject.

Treg cells comprising the CARs of this disclosure can express these CARs and can be used in the methods described herein to treat autoimmune or inflammatory diseases (e.g., rheumatoid arthritis or hidradenitis suppurativa). In some embodiments, the autoimmune or inflammatory disease is rheumatoid arthritis. In some embodiments, the autoimmune or inflammatory disease is hidradenitis suppurativa.

In some embodiments, the administering comprises: (a) isolating T cells from a biological sample obtained from the individual; (b) enriching the T cells for Tregs; (c) transfecting the enriched Treg cells with an expression vector comprising an expression control sequence operatively linked to a nucleotide sequence encoding a chimeric antigen receptor (CAR) comprising an antigen binding domain that binds to a citrullinated polypeptide or citrullinated peptide; (d) expanding the transfected Treg cells; and (e) administering a composition comprising the expanded Treg cells to the individual. In some embodiments, the composition comprising the expanded Treg cells is administered intravenously. In some embodiments, the CAR is any CAR described in Section V.A. above.

Also, the proteins, such as the antibodies (e.g., the antibodies targeting epitopes of citrullinated polypeptides or citrullinated polypeptides), produced by a transformed/recombinant host cell can be purified according to any suitable method. Such methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence and glutathione-S-transferase can be attached to the protein to allow easy purification by passage over an appropriate affinity column. In some embodiments, proteins can also be physically characterized using such techniques as proteolysis, high performance liquid chromatography (HPLC), nuclear magnetic resonance and x-ray crystallography.

### C. Compositions

Also disclosed are pharmaceutical compositions comprising a cell comprising the disclosed CAR and a pharmaceutically acceptable carrier.

As used herein, the term “pharmaceutical composition” refers to a composition comprising a pharmaceutical compound (e.g., a drug or a recombinant Treg cell as described herein) and a pharmaceutically acceptable carrier.

As used herein, the term “pharmaceutically acceptable” refers to a carrier that is compatible with the other ingredients of a pharmaceutical composition and can be safely administered to an individual. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

Pharmaceutical compositions can be formulated for any route of administration, including mucosal (e.g., nasal, sublingual, vaginal, buccal, or rectal), parenteral (e.g., subcutaneous, intravenous, intramuscular, or intraarterial injection, either bolus or infusion), oral, or transdermal.

Injectable (e.g., intravenous) compositions can comprise a solution of the composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. The formulations of compositions can be presented in unit-dose or multi-dose sealed containers, such as ampoules and vials.

Cells can be cryopreserved. Cryopreservation can include formulating cells with a cryopreservation agent, such as DMSO. Commercially available media include, for example, CryoStor® and pZerve®, available from Millipore Sigma.

Compositions can be formulated as dosage forms for administration. The term “dosage form” refers to the particular format of the pharmaceutical and depends on the route of administration. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors or based on therapeutic progress.

The pharmaceutical preparation can be packaged or prepared in unit dosage form. In such form, the preparation is subdivided into unit doses containing appropriate quantities of the active component, e.g., according to the dose of the therapeutic agent or concentration of the composition. The unit dosage form can be a packaged preparation, the package containing discrete quantities of preparation. The composition can, if desired, also contain other compatible therapeutic agents.

### D. Administration

An exemplary method of this disclosure includes isolating T lymphocytes from a biological sample obtained from the individual. Such T cells can be isolated by immunoaffinity, for example, using solid supports derivatized and anti-CD4 antibodies. CD4+T regulatory cells (Treg) can be separated from non-Treg cells based on their marker profile. Treg cells are CD4+, CD25+, CD127lo. Non-Treg cells include: CD4+, CD25+ and CD127+. The isolated T reg cells are then transfected with an expression vector encoding a chimeric antigen receptor (CAR) of this disclosure. The transfected cells are expanded. The expanded cells are administered to the individual.

The pharmaceutical composition can be administered by any suitable route, including but not limited to intravenous, subcutaneous, intramuscular or intraperitoneal routes. An example of administration of a pharmaceutical composition includes storing the composition at 10 mg/ml in sterile isotonic aqueous saline solution for injection at 4° C., and diluting it in either 100 ml or 200 ml 0.9% sodium chloride for injection prior to administration to the patient. The pharmaceutical composition is administered by intravenous infusion over the course of 1 hour at a dose of between 0.2 and 10 mg/kg. In other embodiments, the pharmaceutical composition is administered by intravenous infusion over a period of between 15 minutes and 2 hours. In still other embodiments, the administration procedure is via sub-cutaneous bolus injection.

The dose of the composition is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the composition (e.g., half-life of the composition in the circulation) and the pharmacodynamic response (e.g., the duration of the therapeutic effect of the composition). In some embodiments, the in vivo half-life of between about 7 and about 25 days, and composition dosing is repeated between once per week and once every 3 months.

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Thus, in some embodiments, additional administration is dependent on patient progress, e.g., the patient is monitored between administrations. For example, after the first administration or round of administrations, the patient can be monitored for rate of tumor growth, recurrence (e.g., in the case of a post-surgical patient), or general disease-related symptoms such as weakness, pain, nausea, etc.

In certain embodiments, the T cells described herein are administered to the synovia of the joints of subjects having rheumatoid arthritis. Such administration can be by injection directly into the joint.

In certain embodiments, the T cells described herein can be administered intravenously to subjects having hidradenitis suppurativa. In other embodiments, the T cells described herein can be administered via intradermal injection to subjects having hidradenitis suppurativa. Such administration can be by injection or infusion directly into the dermis.

### VI. Method of Monitoring Treatment

In some embodiments, the methods provided herein are used to monitor the efficacy of a cell therapy treatment by detecting the level or presence of a citrullinated polypeptide or a citrullinated peptide, wherein a reduction of the level of the citrullinated polypeptide or citrullinated peptide indicates that the cell therapy is effective. In some embodiments, the therapeutic regimen is altered based upon results of the assays provided herein, for example therapy is continued or discontinued. In some embodiments, the cell therapy is for use in the treatment of an autoimmune or inflammatory disease. In some embodiments, the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease. In some embodiments, the autoimmune or inflammatory disease is rheumatoid arthritis or hidradenitis suppurative.

Provided herein are methods of monitoring treatment of an autoimmune or inflammatory disease (e.g., rheumatoid arthritis or hidradenitis suppurativa) in an individual comprising a) detecting the presence or amount of a citrullinated polypeptide or citrullinated peptide in a biological sample obtained from the individual receiving treatment for the autoimmune or inflammatory disease (e.g., rheumatoid arthritis or hidradenitis suppurativa); and b) comparing the presence or amount of the citrullinated polypeptide or citrullinated peptide in the biological sample to a biological sample obtained from the individual prior to receiving treatment for the autoimmune or inflammatory disease (e.g., rheumatoid arthritis or hidradenitis suppurativa) or from an earlier time point during treatment, wherein a reduction in the amount of citrullinated polypeptides or citrullinated peptides in a biological sample compared to the amount of citrullinated polypeptides or citrullinated peptides in a biological sample prior to receiving treatment for the autoimmune or inflammatory disease (e.g., rheumatoid arthritis or hidradenitis suppurativa) or from an earlier time point during treatment is indicative of the effective treatment for the autoimmune or inflammatory disease (e.g., rheumatoid arthritis or hidradenitis suppurativa) in the individual. In some embodiments, the detecting comprises any of the methods of detecting described in Section II above.

In some embodiments, provided herein is a method of monitoring treatment of rheumatoid arthritis in an individual comprising a) detecting the presence or amount of a citrullinated polypeptide or citrullinated peptide in a biological sample obtained from the individual receiving treatment for rheumatoid arthritis; and b) comparing the presence or amount of the citrullinated polypeptide or citrullinated peptide in the biological sample to a biological sample obtained from the individual prior to receiving treatment for rheumatoid arthritis or from an earlier time point during treatment, wherein a reduction in the amount of citrullinated polypeptides or citrullinated peptides in a biological sample compared to the amount of citrullinated polypeptides or citrullinated peptides in a biological sample prior to receiving treatment for rheumatoid arthritis or from an earlier time point during treatment is indicative of the effective treatment for rheumatoid arthritis in the individual.

In some embodiments, provided herein is a method of monitoring treatment of hidradenitis suppurativa in an individual comprising a) detecting the presence or amount of a citrullinated polypeptide or citrullinated peptide in a biological sample obtained from the individual receiving treatment for hidradenitis suppurativa; and b) comparing the presence or amount of the citrullinated polypeptide or citrullinated peptide in the biological sample to a biological sample obtained from the individual prior to receiving treatment for hidradenitis suppurativa or from an earlier time point during treatment, wherein a reduction in the amount of citrullinated polypeptides or citrullinated peptides in a biological sample compared to the amount of citrullinated polypeptides or citrullinated peptides in a biological sample prior to receiving treatment for hidradenitis suppurativa or from an earlier time point during treatment is indicative of the effective treatment for hidradenitis suppurativa in the individual.

The methods of monitoring treatment can occur at any time during treatment or after termination of treatment. Once a patient has initiated treatment, a biological sample (such as any of the biological samples described herein) can be obtained from the individual at any time points of interest. These time points are typically selected by a healthcare provider. In some embodiments, time points at which a biological sample can be obtained from the individual being treated include about any one of 1 week, 2 weeks, 3 weeks, 4 weeks, 5 weeks, 6 weeks, 7 weeks, 8 weeks, 8 weeks, 10 weeks, 11 weeks, 12 weeks, 13 weeks, 14 weeks, 15 weeks, 16 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 1 year, 2 years, 3 years, 4 years, 5 years, 6 years, 7 years, 8 years, 9 years, 10 years or more.

In some embodiments, time points at which a biological sample (such as any of the biological samples described herein) can be obtained from the individual after termination of treatment include about any one of 1 week, 2 weeks, 3 weeks, 4 weeks, 5 weeks, 6 weeks, 7 weeks, 8 weeks, 8 weeks, 10 weeks, 11 weeks, 12 weeks, 13 weeks, 14 weeks, 15 weeks, 16 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 1 year, 2 years, 3 years, 4 years, 5 years, 6 years, 7 years, 8 years, 9 years, 10 years or more.

In some embodiments, time points at which a biological sample (such as any of the biological samples described herein) can be obtained from the individual after administration of a cell therapy (for example, a CAR T cell therapy, such as any described in the present disclosure) include about any one of 1 week, 2 weeks, 3 weeks, 4 weeks, 5 weeks, 6 weeks, 7 weeks, 8 weeks, 8 weeks, 10 weeks, 11 weeks, 12 weeks, 13 weeks, 14 weeks, 15 weeks, 16 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 1 year, 2 years, 3 years, 4 years, 5 years, 6 years, 7 years, 8 years, 9 years, 10 years or more.

In some cases, the method of monitoring treatment of an autoimmune or inflammatory disease (e.g., rheumatoid arthritis or hidradenitis suppurativa) can include any currently known, proposed, purported, or hypothesized treatment of the autoimmune or inflammatory disease (e.g., rheumatoid arthritis or hidradenitis suppurativa), including any future treatments for the autoimmune or inflammatory disease (e.g., rheumatoid arthritis or hidradenitis suppurativa). In some embodiments, the method of monitoring treatment of an autoimmune or inflammatory disease (e.g., rheumatoid arthritis or hidradenitis suppurativa) further comprises re-administration of the current therapy, termination of the current therapy and initiation of a new therapy, re-administration of the current therapy and initiation of a new, concomitant therapy, etc. In some embodiments, the method further comprises administration of a cell therapy comprising a T cell comprising a chimeric antigen receptor (CAR) that binds to a citrullinated polypeptide or citrullinated peptide, as described in the present disclosure.

### VII. Kits

As used herein, the term “kit” refers to a collection of items intended for use together. The kit can optionally include a reference agent and/or instructions for use thereof. A kit can further include a shipping container adapted to hold a container, such as a vial, that contains a composition as disclosed herein. A kit can include a container that contains within it the collection of items.

Kits of this disclosure can comprise a capture antibody and a detection antibody as described herein, and instructions for use, which can be sterilely packaged or otherwise contained in a container, such as a bag or box. Also included in the kit can be one or more sample plates, such as a 96-well or 384 well sample plate. In some embodiments, the kit can further comprise beads for affixing or otherwise immobilizing the capture antibody included in the kit. In some embodiments, the kit can be used in any of the methods described herein for the detection of the presence or amount of a citrullinated polypeptide or citrullinated peptide as described herein.

### EXAMPLES

### Example 1: An Antibody-Based Detection Assay and a Cell-Based Assay for the Detection of Citrullinated Antigen

Citrullination is a well-characterized component of rheumatoid arthritis (RA) disease etiology. Citrullinated proteins accumulate in the synovial fluid in the joints of patients with RA, and anti-citrullinated protein antibodies (ACPAs) are observed in majority of patients. ACPAs recognize many citrullinated epitopes, although the specific epitopes differ between patients. Described herein is an assay for determining the level of citrullination in an individual.

An antibody that binds to citrullinated polypeptides comprising the VH and VL sequences set forth in Table 1 was used as a capture antibody that was immobilized to the bottom of the sample wells of a sample plate.

Table 1. Anti-citrullinated polypeptide antibody VH and VL sequences.

TABLE 1
Anti-citrullinated polypeptide antibody VH and VL
sequences.

| Antibody | Amino Acid | SEQ ID | Nucleic Acid | SEQ ID |
|---|---|---|---|---|
| domain | sequence | NO: | sequence | NO: |
| CV VH-#1 | HLHLQESGPGLVKPSE | 7 | CACCTGCACTTGCAGGA | 17 |
| | TLSLTCTVSGGSINDT | | GTCGGGCCCAGGACTTG | |
| | TYYWGWIRQPPGKGL | | TGAAGCCTTCGGAGACC | |
| | EWIGSIYYRGNTHYN | | CTGTCCCTCACCTGCACT | |
| | SSLRSRVTMSVDTSK | | GTCTCTGGTGGCTCCATC | |
| | NRFSLKVTSVTAADT | | AACGATACCACTTACTAC | |
| | AVYYCARLDPFDYW | | TGGGGCTGGATTCGCCA | |
| | GRGTLVTVSS | | GCCCCCCGGGAAGGGAC | |
| | | | TGGAGTGGATTGGGAGT | |
| | | | ATCTATTACCGGGGGAA | |
| | | | CACCCACTACAATTCGTC | |
| | | | CCTGAGGAGTCGCGTCA | |
| | | | CCATGTCTGTCGACACTT | |
| | | | CCAAGAACCGATTCTCCC | |
| | | | TGAAGGTCACTTCTGTGA | |
| | | | CTGCCGCAGACACGGCT | |
| | | | GTCTATTACTGTGCGAGA | |
| | | | CTCGACCCATTTGACTAC | |
| | | | TGGGGCCGTGGCACCCT | |
| | | | GGTCACTGTCTCGAGC | |
| CV VL #2 | SYVLTQPPSVSVAPGK | 10 | TCCTATGTGCTGACTCAG | 20 |
| | TARITCGGNNIGSKSV | | CCACCCTCAGTGTCAGTG | |
| | HWYQQKPGQAPVLVI | | GCCCCAGGAAAGACGGC | |
| | YYDSDRPSGIPERFSG | | CAGGATTACCTGTGGGG | |
| | SNSGNTATLTISRVEA | | GAAACAACATTGGAAGT | |
| | GDEADYYCQVWDSS | | AAAAGTGTGCACTGGTA | |
| | SDHQVFGTGTKVTVL | | CCAGCAGAAGCCAGGCC | |
| | | | AGGCCCCTGTGCTGGTCA | |
| | | | TCTATTATGATAGCGACC | |
| | | | GGCCCTCAGGGATCCCT | |
| | | | GAGCGATTCTCTGGCTCC | |
| | | | AACTCTGGGAACACGGC | |
| | | | CACCCTGACCATCAGCA | |
| | | | GGGTCGAAGCCGGGGAT | |
| | | | GAGGCCGACTATTACTGT | |
| | | | CAGGTGTGGGACAGTAG | |
| | | | TAGTGATCACCAAGTCTT | |
| | | | CGGAACTGGGACCAAGG | |
| | | | TCACCGTCCTA | |

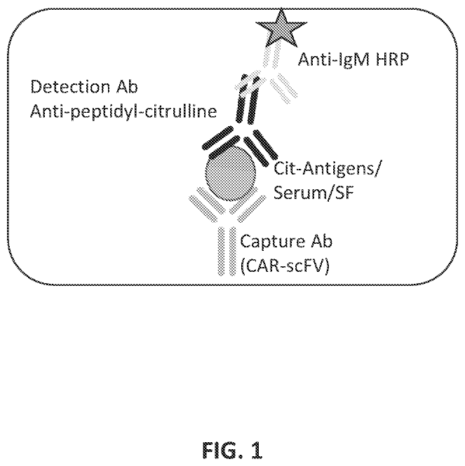

FIG. 1 shows the detection assay setup. In brief, the capture antibody was affixed to the sample wells of a sample plate. The sample well of the plate were washed and blocked according to standard blocking protocol. Serum or synovial fluid obtained from either healthy controls or patients with RA were incubated in the sample well of the plate.

The sample wells then were washed to remove the supernatant. The commercially available anti-peptidyl-citrulline antibody clone F95 was used as the detection antibody. The F95 anti-peptidyl-citrulline antibody binds to a different epitope of citrullinated polypeptides than the capture antibody described above.

The sample wells were then washed again prior to incubation with an IgM secondary antibody conjugated to HRP that binds to the Fc region of the F95 detection antibody. The HRP substrate TMB (3,3′,5,5′-tetramethylbenzidine) was added for detection of HRP levels, followed by incubation with a sulfuric or phosphoric acid stopping solution. Results were analyzed using a microplate reader with a 450 nm filter to read OD450 value in comparison to a standard curve.

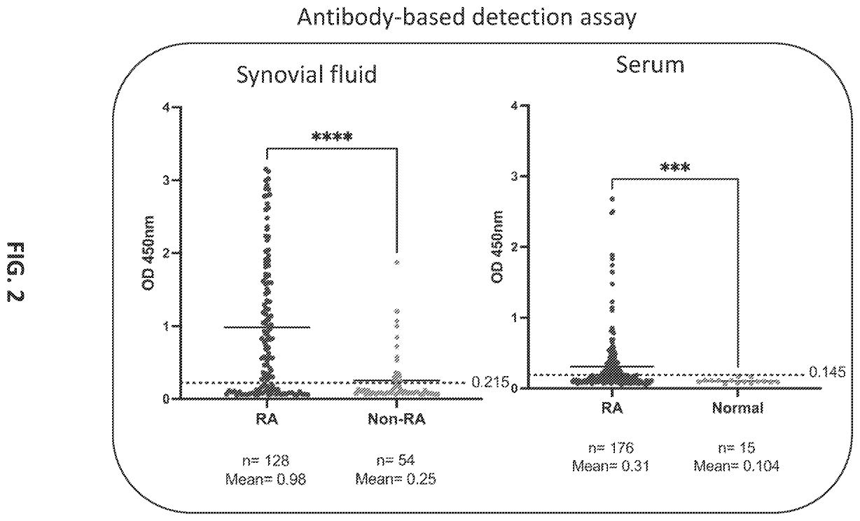

Citrullinated polypeptide levels were found to be significantly elevated in patients with RA compared to healthy controls using this detection assay. Un-matched patient samples from synovial fluid (n=176 RA, n=54 non-RA) demonstrated 68% positivity among patients with RA compared to samples from serum (n=130 RA, n=15 non-RA), which demonstrated 50% positivity among patients with RA, as shown in FIG. 2. Elevated protein citrullination was detected in 84% of synovial fluid samples and in 48% of serum samples from patient-matched synovial fluid and serum samples obtained from patients with active RA disease (n=50; data not shown; see Table 2 below for patient demographics). Furthermore, overall levels of citrullinated polypeptides were also found to be higher in synovial fluid than in serum (RA patients: SF mean=0.98 vs. Serum mean=0.31; Healthy controls: SF mean=0.25 vs. Serum mean=0.104).

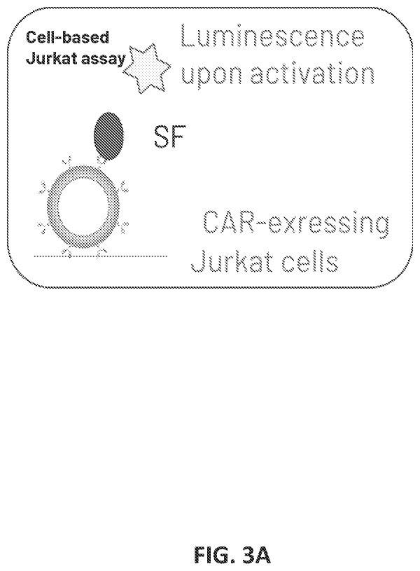

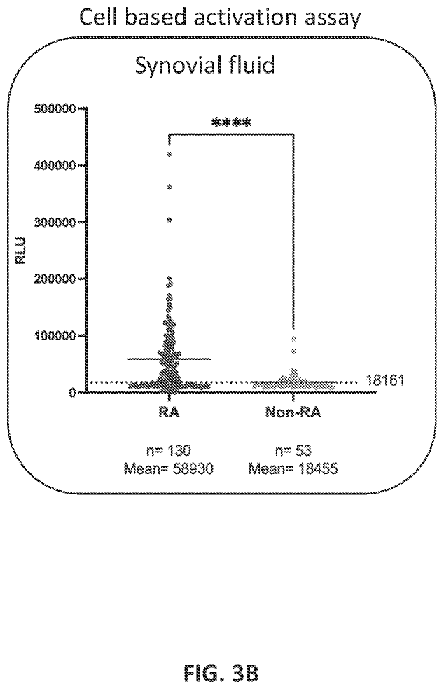

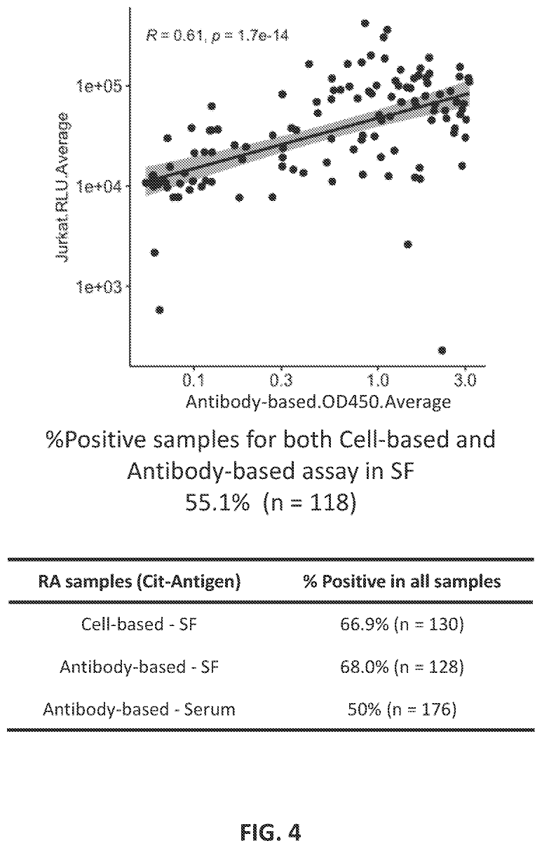

Results from this novel detection assay were then compared to a cell-based Jurkat assay (see FIG. 3A), where the Jurkat cells express a chimeric antigen receptor (CAR) that includes an antigen-binding domain with the same VH and VL sequences as the capture antibody described above (CV-CAR). CV-CAR-Jurkat reporter cells were then incubated with synovial fluid or serum obtained from RA patients or healthy controls. In these cells, activation of the CAR by binding to citrullinated polypeptides causes emission of a luminescent signal that can be by a plate reader and reported as relative light unit (RLU). FIG. 3B shows significantly increased CAR activation in RA patients compared to healthy controls. The levels of cell activation were higher by 4.6-fold in synovial fluid compared to serum (data not shown). A dot plot showing the correlation between cell-based assay and antibody detection assay results, wherein each dot represents one patent sample, is shown in FIG. 4. FIG. 4 shows that there is positive correlation (n=50, Spearman R=0.48, p<0.05) between the levels of citrullinated polypeptides in RA patients and CAR activation using the CV-CAR-Jurkat reporter cell assay.

The results demonstrate that both an antibody-based assay and a cell-based assay can be used to detect citrullinated polypeptides in synovial fluid and serum. The highest sensitivity was identified using the antibody detection assay on synovial fluid samples.

### Example 2: Comparison of Antibody-Based Assay for Detection of Citrullinated Antigen and Other Methods of Detecting RA in Patient Samples

Patient-matched synovial fluid and serum samples were obtained from RA patients and healthy controls. Demographics of the RA patients are provided in Table 2 below. The levels of citrullinated polypeptide detected in synovial fluid of patients was compared to other potential indicators of RA, including C reactive protein (CRP) and inflammatory cytokines.

Table 2. RA patient demographics.

| | Overall (N = 50) | |
|---|---|---|
| Gender | | |
| Male | 10 | (20.0%) |
| Female | 39 | (78.0%) |
| Missing | 1 | (2.0%) |
| Disease Duration | | |
| Mean (SD) | 11.5 | (10.1) |
| Median [Min, Max] | 9.50 | [0, 44.0] |
| Missing | 10 | (20.0%) |
| Clinical CCP | | |
| Positive | 36 | (72.0%) |
| Negative | 10 | (20.0%) |
| Missing | 4 | (8.0%) |
| Clinical RF | | |
| Positive | 28 | (56.0%) |
| Negative | 12 | (24.0%) |
| Missing | 10 | (20.0%) |
| Current Treatment | | |
| JAKi | 1 | (2.0%) |
| MTX | 5 | (10.0%) |
| MTX/Predn | 5 | (10.0%) |
| CTLA4-IgG | 1 | (2.0%) |
| LEF/CTLA4-IgG | 1 | (2.0%) |
| MTX/Predn/TNFi | 2 | (4.0%) |
| MTX/SSZ | 2 | (4.0%) |
| MTX/TNFi | 6 | (12.0%) |
| Predn | 4 | (8.0%) |
| No Tx | 6 | (12.0%) |
| MTX/SSZ/TNFi | 1 | (2%) |
| MTX/HCQ | 1 | (2.0%) |
| SSZ | 2 | (4.0%) |
| TNFi/Predn | 2 | (4.0%) |
| Missing | 11 | (22.0%) |

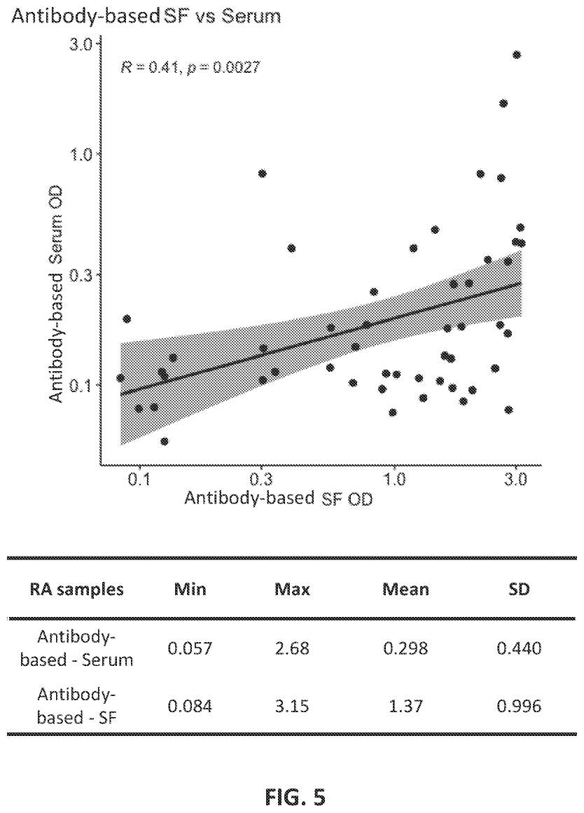

FIG. 5 shows a positive correlation (n=50, Spearman R=0.41, p<0.05) between citrullinated polypeptide levels in patient sample-matched synovial fluid and serum as tested by the antibody detection assay. Although sensitivity to and overall level of citrullinated polypeptides were higher in synovial fluid, results from serum samples were still predictive of RA.

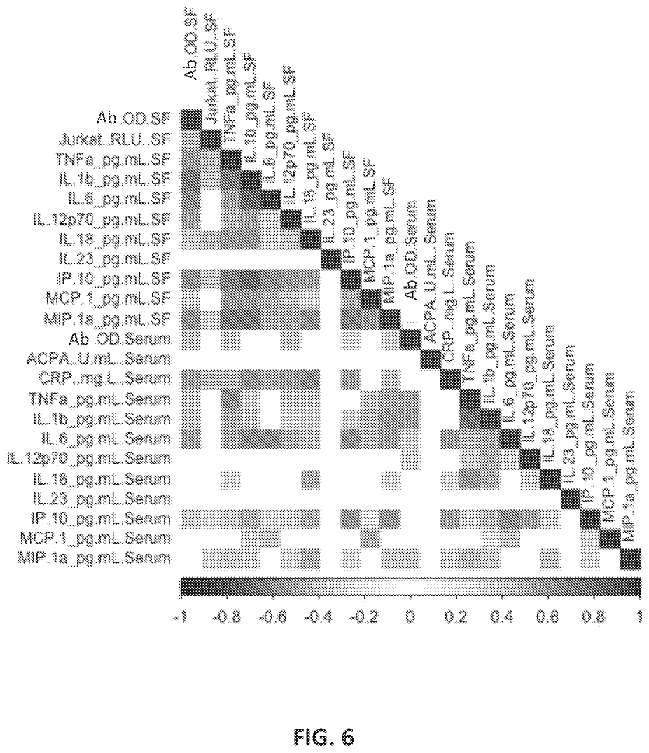

Citrullinated antigen levels in synovial fluid are correlated with systemic markers of inflammation, including CRP and IL-6, TNF-α, IL-1β, and IP-10. These correlations are shown in a heat map in FIG. 6. Synovial fluid levels of citrullinated antigen were positively and significantly correlated with all variables displayed with shades of blue color. These include 1) the SF-tested cell-based assay described in Example 1 above, TNF-α, IL-1β, IL-6, IL-12p70, IL-18, IP-10, MCP-1, and MIP-1a, and 2) the serum-tested citrullinated-polypeptide-antibody detection assay, CRP, TNF-α, IL-1β, IL-6, and IP-10.

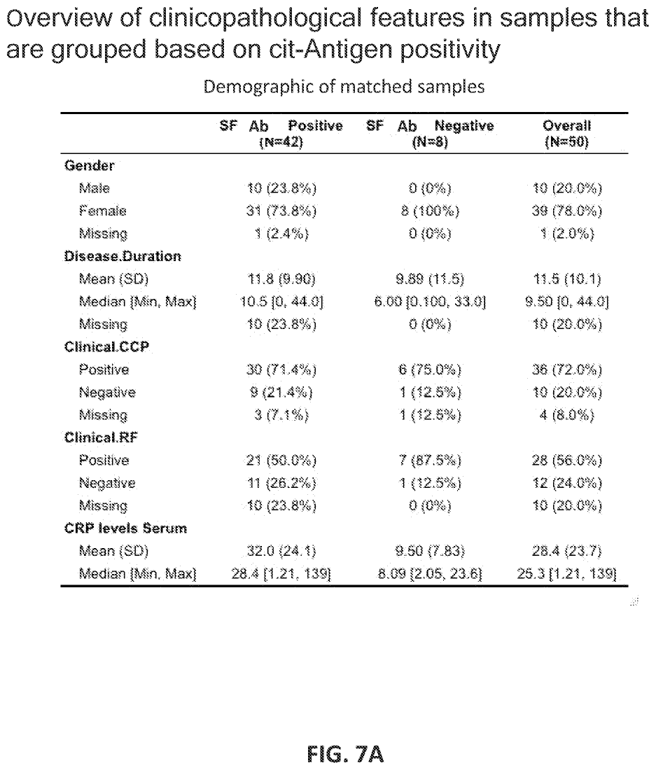

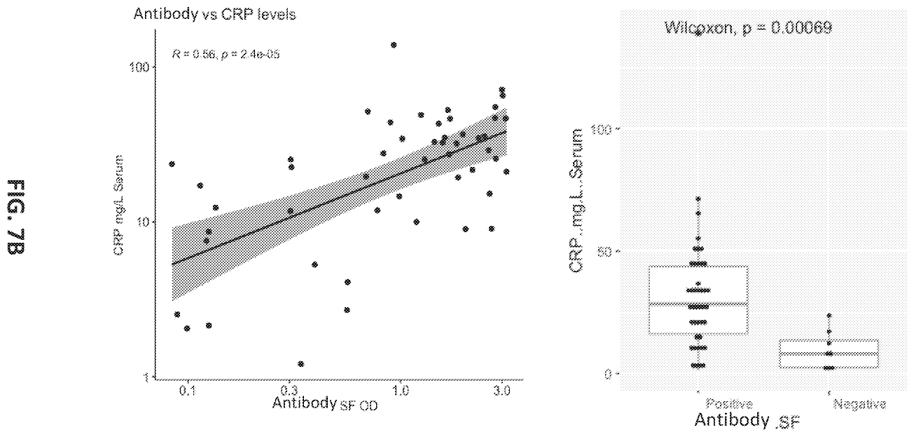

Samples were further assessed for a correlation between antibody-detected citrullinated polypeptide levels and serum CRP levels. Clinicopathological features of samples grouped by synovial fluid citrullinated polypeptide positivity showed a clear increase in serum CRP levels compared to the patients that were negative for synovial fluid citrullinated polypeptide (see FIG. 7A). Serum CRP levels as detected by ELISA showed a positive correlation (n=50, Spearman R=0.56, p<0.05) to synovial fluid citrullinated polypeptide. This correlation is displayed by linear regression and box plot in FIG. 7B.

The primary biologic markers deemed clinically useful in patients with RA include: rheumatoid factors (RF), anti-cyclic citrullinated peptide (anti-CCP) antibodies, erythrocyte sedimentation rate (ESR), and C-reactive protein (CRP). The results from the comparison tests described above confirm that this novel antibody detection assay can be used to identify the presence of citrullinated polypeptides with high sensitivity in various types of biological samples that can be used to diagnose RA and identify patients suitable for treatment, in particular with a cell comprising a CAR that binds to a citrullinated polypeptide.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### EXEMPLARY EMBODIMENTS

1. A method of detecting the presence or amount of a citrullinated polypeptide or citrullinated peptide in a biological sample obtained from an individual comprising
   a) incubating the biological sample with a capture antibody that binds to the citrullinated polypeptide or the citrullinated peptide to form a complex comprising the capture antibody and the citrullinated polypeptide or citrullinated peptide;
   b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody, thereby detecting the presence or amount of the citrullinated polypeptide or citrullinated peptide.
2. A method of diagnosing rheumatoid arthritis in an individual comprising
   a) incubating a biological sample obtained from the individual with a capture antibody that binds to a citrullinated polypeptide or citrullinated peptide to form a complex comprising the capture antibody and the citrullinated polypeptide or citrullinated peptide;
   b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with rheumatoid arthritis based upon the presence or amount of the citrullinated polypeptide or citrullinated peptide.
3. A method of selecting an individual having rheumatoid arthritis for treatment with a T cell therapy comprising a T cell comprising a chimeric antigen receptor (CAR) that binds to a citrullinated polypeptide or citrullinated peptide comprising
   a) detecting the presence or amount of the citrullinated polypeptide or citrullinated peptide in a biological sample obtained from the individual; and
   b) selecting the individual for treatment with the T cell therapy if the biological sample comprises citrullinated polypeptides or citrullinated peptides or an amount of citrullinated polypeptides or citrullinated peptides that is above a threshold or a reference level.
4. The method of Embodiment 3, further comprising comparing the amount of the citrullinated polypeptide or citrullinated peptide in the biological sample to a reference level or reference sample.
5. The method of Embodiment 4, wherein the reference sample is a biological sample obtained from an individual not having rheumatoid arthritis.
6. A method of treating rheumatoid arthritis in an individual comprising
   i) detecting a citrullinated polypeptide or citrullinated peptide in a biological sample obtained from an individual comprising
      a) incubating the biological sample with a capture antibody that binds to a citrullinated polypeptide or citrullinated peptide to form a complex comprising the capture antibody and the citrullinated polypeptide or citrullinated peptide;
      b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
      c) detecting the complex comprising the capture antibody, the citrullinated polypeptide or the citrullinated peptide, and the detection antibody; and
   ii) administering to the individual an effective amount of a composition comprising a T cell comprising a chimeric antigen receptor (CAR) comprising an antigen binding domain that specifically binds to a citrullinated polypeptide or citrullinated peptide, if the biological sample comprises citrullinated polypeptides or citrullinated peptides or an amount of citrullinated polypeptides or citrullinated peptides that is above a threshold or a reference level.
7. A method of monitoring treatment of rheumatoid arthritis in an individual comprising
   a) detecting the presence or amount of a citrullinated polypeptide or citrullinated peptide in a biological sample obtained from the individual receiving treatment for rheumatoid arthritis; and
   b) comparing the presence or amount of the citrullinated polypeptide or citrullinated peptide in the biological sample to a biological sample obtained from the individual prior to receiving treatment for rheumatoid arthritis or from an earlier time point during treatment,
   wherein a reduction in the amount of citrullinated polypeptides or citrullinated peptides in a biological sample compared to the amount of citrullinated polypeptides or citrullinated peptides in a biological sample prior to receiving treatment for rheumatoid arthritis or from an earlier time point during treatment is indicative of the effective treatment for rheumatoid arthritis in the individual.
8. A method of diagnosing rheumatoid arthritis in an individual comprising
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
   b) detecting the signal emitted by the CAR to determine the presence or amount of the citrullinated polypeptide or citrullinated peptide, wherein the individual is diagnosed with rheumatoid arthritis based upon the presence or amount of the citrullinated polypeptide or citrullinated peptide.
9. A method of treating rheumatoid arthritis in an individual comprising
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated;
   b) detecting the signal emitted by the CAR to determine the presence or amount of the citrullinated polypeptide or citrullinated peptide; and
   c) administering to the individual an effective amount of a composition comprising a T cell comprising a chimeric antigen receptor (CAR) comprising an antigen binding domain that specifically binds to a citrullinated polypeptide or citrullinated peptide if the biological sample comprises citrullinated polypeptides or citrullinated peptides or an amount of citrullinated polypeptides or citrullinated peptides that is above a threshold or a reference level.
10. The method of Embodiment 3 or Embodiment 7, wherein the detecting comprises
   a) incubating the biological sample with a capture antibody that binds to the citrullinated polypeptide or citrullinated peptide to form a complex comprising the capture antibody and the citrullinated polypeptide or citrullinated peptide;
   b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody.
11. The method of Embodiment 3 or Embodiment 7, wherein the detecting comprises:
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
   b) detecting the signal emitted by the CAR to determine the presence or amount of the citrullinated polypeptide or citrullinated peptide.
12. The method of any one of Embodiments 1-11, wherein the biological sample is serum and/or synovial fluid.
13. The method of any one of Embodiments 1-12, wherein the biological sample is serum.
14. The method of any one of Embodiments 1-12, wherein the biological sample is synovial fluid.
15. The method of Embodiment 12, wherein the serum and the synovial fluid are from the same individual.
16. The method of any one of Embodiments 1, 2, 6, 10, and 12-15, wherein the capture antibody is immobilized to a sample plate.
17. The method of any one of Embodiments 1, 2, 6, 10, and 12-15, wherein the capture antibody is immobilized to a bead.
18. The method of any one of Embodiments 1, 2, 6, 10, and 12-17, wherein the detection antibody is conjugated to a label molecule.
19. The method of any one of Embodiments 1, 2, 6, 10, and 12-18, wherein the detection antibody is detected by the binding of a secondary antibody, wherein the secondary antibody is conjugated to a label molecule.
20. The method of Embodiment 19, wherein the secondary antibody binds to a constant region of the detection antibody.
21. The method of Embodiment 20, wherein the secondary antibody is an anti-IgM antibody or fragment thereof.
22. The method of any one of Embodiments 18-21, wherein the label molecule is selected from the group consisting of a chemiluminescent enzyme, a fluorescent tag, and a radiolabeled tag.
23. The method of any one of Embodiments 18-22, wherein the label molecule is a horseradish peroxidase enzyme or an alkaline phosphatase enzyme.
24. The method of Embodiment 23, wherein the horseradish peroxidase enzyme or alkaline phosphatase enzyme bind a substrate molecule, wherein the substrate molecule is converted into a detectable product.
25. The method of Embodiment 24, wherein the substrate molecule is colorimetric/chromogenic, fluorescent, chemifluorescent, or chemiluminescent.
26. The method of any one of Embodiments 1, 2, 6, 10, and 12-25, wherein the capture antibody and the detection antibody bind to different epitopes of the citrullinated polypeptide or citrullinated peptide.
27. The method of Embodiment 26, wherein the capture antibody and/or the detection antibody binds to one or more of: citrullinated vimentin, citrullinated filaggrin, citrullinated fibrinogen, citrullinated tenascin C, and/or citrullinated fragments thereof.
28. The method of Embodiment 27, wherein the capture antibody and/or the detection antibody binds to two or more of citrullinated vimentin, citrullinated filaggrin, citrullinated fibrinogen, citrullinated tenascin C, and/or citrullinated fragments thereof.
29. The method of Embodiment 27 or Embodiment 28, wherein the capture antibody or the detection antibody is an anti-peptidyl-citrulline antibody.
30. The method of Embodiment 29, wherein the anti-peptidyl-citrulline antibody is the F95 antibody clone.
31. The method of any one of Embodiments 1, 2, 6, 10, and 12-30, wherein the capture antibody or the detection antibody comprises an antibody fragment selected from the group consisting of a Fv, scFv, camelid nanobody, heavy chain only antibody, full-length antibody, and aptamer.
32. The method of Embodiment 31, wherein the capture antibody or the detection antibody comprises a full-length antibody.
33. The method of Embodiment 32, wherein the capture antibody comprises an Fc domain.
34. The method of any one of Embodiments 1, 2, 6, 10, and 12-33, wherein the capture antibody, or the detection antibody, and/or the CAR comprises a VH and a VL, wherein:
   (i) the VH comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and
   (ii) the VL comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6.
35. The method of Embodiment 34, wherein the capture antibody, the detection antibody, or the CAR comprises a VH comprising the amino acid sequence of SEQ ID NO:7 or 8 and/or a VL comprising the amino acid sequence of SEQ ID NO:9 or 10.
36. The method of Embodiment 35, wherein the VH comprises the amino acid sequence of SEQ ID NO:7 and the VL comprises the amino acid sequence of SEQ ID NO:10.
37. The method of any one of Embodiments 1-36, further comprising:
   a) detecting the level of one or more proteins selected from the group consisting of C reactive protein (CRP), IL-1β, IL-6, IL-18, IL-12p70, IP-10, MCP-1, MIP-1a, and TNF-α in a biological sample;
   b) comparing the level of one or more proteins selected from the group consisting of C reactive protein (CRP), IL-1β, IL-6, IL-18, IL-12p70, IP-10, MCP-1, MIP-1a, and TNF-α to a reference level; and
   c) selecting the individual as having rheumatoid arthritis when the level of one or more proteins selected from the group consisting of C reactive protein (CRP), IL-1β, IL-6, IL-18, IL-12p70, IP-10, MCP-1, MIP-1a, and TNF-α in the biological sample is above the reference level.
38. The method of Embodiment 6, wherein the administering comprises:
   (a) isolating T cells from a biological sample obtained from the individual;
   (b) enriching the T cells for Tregs;
   (c) transfecting the enriched Treg cells with an expression vector comprising an expression control sequence operatively linked to a nucleotide sequence encoding a chimeric antigen receptor (CAR) comprising an antigen binding domain that binds to a citrullinated polypeptide or citrullinated peptide;
   (d) expanding the transfected Treg cells; and
   (e) administering the expanded Treg cells to the individual.
39. The method of any one of Embodiments 1-38, wherein the individual is a human.
40. The method of any one of Embodiments 6, 38, and 39, wherein the Treg is administered intravenously.
41. The method of any one of Embodiments 3, 4,7-9, and 11-40, wherein the CAR comprises an optional signal peptide, a CAR antigen binding domain, an optional hinge domain, a transmembrane domain, one or more optional co-stimulatory domains, and an intracellular signaling domain, wherein the antigen binding domain specifically binds to a citrullinated polypeptide or citrullinated peptide.
42. The method of Embodiment 41, wherein the CAR antigen binding domain binds to a citrullinated peptide or citrullinated fragment thereof.
43. The method of Embodiment 41 or Embodiment 42, wherein the CAR antigen binding domain binds to one or more of: citrullinated vimentin, citrullinated filaggrin, citrullinated fibrinogen, citrullinated tenascin C, or citrullinated fragments thereof.
44. The method of any one of Embodiments 41-43, wherein the CAR antigen binding domain comprises an antibody fragment selected from the group consisting of an Fv, scFv, camelid nanobody, heavy chain only antibody, or aptamer.
45. The method of any one of Embodiments 41-44, wherein the CAR antigen binding domain comprises an scFv.
46. The method of any one of Embodiments 41-45, wherein the CAR antigen binding domain comprises the same VH and VL as the capture antibody or detection antibody of any one of Embodiments 1-36.
47. The method of Embodiment 45, wherein antigen binding domain of the CAR comprises an scFV comprising a VH and a VL joined by a linker selected from the group consisting of SEQ ID NOs:11-13.
48. The method of Embodiment 47, wherein the antigen binding domain comprises the amino acid sequence of any one of SEQ ID NOs:14-16.
49. The method of any one of Embodiments 41-48, wherein the intracellular signaling domain is derived from CD3-zeta or an Fc receptor.
50. The method of any one of Embodiments 41-49, wherein the CAR comprises at least one co-stimulatory domain derived from the group consisting of: CD3-zeta, FceR1g, Fcg, CD28, 4-1BB, OX40, CD40L, MyD88, CD40, ICOS, RANK/TRANCE-R, CTLA-4, CTLA-4/CD-28 hybrid, DAP10, CD27, and 2B4.
51. The method of any one of Embodiments 41-50, wherein the CAR further comprises a hinge domain.
52. A kit comprising a capture antibody and a detection antibody and instructions for use according to any one of Embodiments 1-51.

### Embodiments B

1B. A method of detecting the presence or amount of a citrullinated polypeptide or citrullinated peptide in a biological sample obtained from an individual comprising
   a) incubating the biological sample with a capture antibody that binds to the citrullinated polypeptide or the citrullinated peptide to form a complex comprising the capture antibody and the citrullinated polypeptide or citrullinated peptide;
   b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody, thereby detecting the presence or amount of the citrullinated polypeptide or citrullinated peptide.
2B. A method of diagnosing an autoimmune or inflammatory disease in an individual comprising
   a) incubating a biological sample obtained from the individual with a capture antibody that binds to a citrullinated polypeptide or citrullinated peptide to form a complex comprising the capture antibody and the citrullinated polypeptide or citrullinated peptide;
   b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with the autoimmune or inflammatory disease based upon the presence or amount of the citrullinated polypeptide or citrullinated peptide.
3B. A method of selecting an individual having an autoimmune or inflammatory disease for treatment with a T cell therapy comprising a T cell comprising a chimeric antigen receptor (CAR) that binds to a citrullinated polypeptide or citrullinated peptide comprising
   a) detecting the presence or amount of the citrullinated polypeptide or citrullinated peptide in a biological sample obtained from the individual; and
   b) selecting the individual for treatment with the T cell therapy if the biological sample comprises citrullinated polypeptides or citrullinated peptides or an amount of citrullinated polypeptides or citrullinated peptides that is above a threshold or a reference level.
4B. The method of Embodiment 3B, further comprising comparing the amount of the citrullinated polypeptide or citrullinated peptide in the biological sample to a reference level or reference sample.
5B. The method of Embodiment 4B, wherein the reference sample is a biological sample obtained from an individual not having an autoimmune or inflammatory disease.
6B. A method of treating an autoimmune or inflammatory disease in an individual comprising
   i) detecting a citrullinated polypeptide or citrullinated peptide in a biological sample obtained from an individual comprising
      a) incubating the biological sample with a capture antibody that binds to a citrullinated polypeptide or citrullinated peptide to form a complex comprising the capture antibody and the citrullinated polypeptide or citrullinated peptide;
      b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
      c) detecting the complex comprising the capture antibody, the citrullinated polypeptide or the citrullinated peptide, and the detection antibody; and
   ii) administering to the individual an effective amount of a composition comprising a T cell comprising a chimeric antigen receptor (CAR) comprising an antigen binding domain that specifically binds to a citrullinated polypeptide or citrullinated peptide, if the biological sample comprises citrullinated polypeptides or citrullinated peptides or an amount of citrullinated polypeptides or citrullinated peptides that is above a threshold or a reference level.
7B. A method of monitoring treatment of an autoimmune or inflammatory disease in an individual comprising
   a) detecting the presence or amount of a citrullinated polypeptide or citrullinated peptide in a biological sample obtained from the individual receiving treatment for the autoimmune or inflammatory disease; and
   b) comparing the presence or amount of the citrullinated polypeptide or citrullinated peptide in the biological sample to a biological sample obtained from the individual prior to receiving treatment for the autoimmune or inflammatory disease or from an earlier time point during treatment,
   wherein a reduction in the amount of citrullinated polypeptides or citrullinated peptides in a biological sample compared to the amount of citrullinated polypeptides or citrullinated peptides in a biological sample prior to receiving treatment for the autoimmune or inflammatory disease or from an earlier time point during treatment is indicative of the effective treatment for the autoimmune or inflammatory disease in the individual.
8B. A method of diagnosing an autoimmune or inflammatory disease in an individual comprising
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
   b) detecting the signal emitted by the CAR to determine the presence or amount of the citrullinated polypeptide or citrullinated peptide, wherein the individual is diagnosed with rheumatoid arthritis based upon the presence or amount of the citrullinated polypeptide or citrullinated peptide.
9B. A method of treating an autoimmune or inflammatory disease in an individual comprising
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated;
   b) detecting the signal emitted by the CAR to determine the presence or amount of the citrullinated polypeptide or citrullinated peptide; and
   c) administering to the individual an effective amount of a composition comprising a T cell comprising a chimeric antigen receptor (CAR) comprising an antigen binding domain that specifically binds to a citrullinated polypeptide or citrullinated peptide if the biological sample comprises citrullinated polypeptides or citrullinated peptides or an amount of citrullinated polypeptides or citrullinated peptides that is above a threshold or a reference level.
10B. The method of Embodiment 3B or Embodiment 7B, wherein the detecting comprises
   a) incubating the biological sample with a capture antibody that binds to the citrullinated polypeptide or citrullinated peptide to form a complex comprising the capture antibody and the citrullinated polypeptide or citrullinated peptide;
   b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
   c) detecting the complex comprising the capture antibody, the citrullinated polypeptide or the citrullinated peptide, and the detection antibody.
11B. The method of Embodiment 3B or Embodiment 7B, wherein the detecting comprises:
   a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
   b) detecting the signal emitted by the CAR to determine the presence or amount of the citrullinated polypeptide or citrullinated peptide.
12B. The method of any one of Embodiments 2B-11B, wherein the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease.
13B. The method of any one of Embodiments 1B-12B, wherein the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, blood, blood plasma, serum, synovial fluid, and any combination thereof.
14B. The method of any one of Embodiments 1B-13B, wherein the biological sample is serum.
15B. The method of any one of Embodiments 1B-13B, wherein the biological sample is synovial fluid.
16B. The method of Embodiment 13B, wherein the biological sample is serum and synovial fluid, and wherein the serum and the synovial fluid are from the same individual.
17B. The method of any one of Embodiments 1B, 2B, 6B, 10B, and 12B-16B, wherein the capture antibody is immobilized to a sample plate.
18B. The method of any one of Embodiments 1B, 2B, 6B, 10B, and 12B-16B, wherein the capture antibody is immobilized to a bead.
19B. The method of any one of Embodiments 1B, 2B, 6B, 10B, and 12B-18B, wherein the detection antibody is conjugated to a label molecule.
20B. The method of any one of Embodiments 1B, 2B, 6B, 10B, and 12B-19B, wherein the detection antibody is detected by the binding of a secondary antibody, wherein the secondary antibody is conjugated to a label molecule.
21B. The method of Embodiment 20B, wherein the secondary antibody binds to a constant region of the detection antibody.
22B. The method of Embodiment 21B, wherein the secondary antibody is an anti-IgM antibody or fragment thereof.
23B. The method of any one of Embodiments 19B-22B, wherein the label molecule is selected from the group consisting of a chemiluminescent enzyme, a fluorescent tag, and a radiolabeled tag.
24B. The method of any one of Embodiments 19B-23B, wherein the label molecule is a horseradish peroxidase enzyme or an alkaline phosphatase enzyme.
25B. The method of Embodiment 24B, wherein the horseradish peroxidase enzyme or alkaline phosphatase enzyme bind a substrate molecule, wherein the substrate molecule is converted into a detectable product.
26B. The method of Embodiment 25B, wherein the substrate molecule is colorimetric/chromogenic, fluorescent, chemifluorescent, or chemiluminescent.
27B. The method of any one of Embodiments 1B, 2B, 6B, 10B, and 12B-26B, wherein the capture antibody and the detection antibody bind to different epitopes of the citrullinated polypeptide or citrullinated peptide.
28B. The method of Embodiment 27B, wherein the capture antibody and/or the detection antibody binds to one or more of: citrullinated vimentin, citrullinated filaggrin, citrullinated fibrinogen, citrullinated tenascin C, and/or citrullinated fragments thereof.
29B. The method of Embodiment 28B, wherein the capture antibody and/or the detection antibody binds to two or more of citrullinated vimentin, citrullinated filaggrin, citrullinated fibrinogen, citrullinated tenascin C, and/or citrullinated fragments thereof.
30B. The method of Embodiment 28B or Embodiment 29B, wherein the capture antibody or the detection antibody is an anti-peptidyl-citrulline antibody.
31B. The method of Embodiment 30B, wherein the anti-peptidyl-citrulline antibody is the F95 antibody clone.
32B. The method of any one of Embodiments 1B, 2B, 6B, 10B, and 12B-31B, wherein the capture antibody or the detection antibody comprises a full-length antibody or an antibody fragment selected from the group consisting of a Fv, scFv, camelid nanobody, heavy chain only antibody, and aptamer.
33B. The method of Embodiment 32B, wherein the capture antibody or the detection antibody comprises a full-length antibody.
34B. The method of Embodiment 33B, wherein the capture antibody comprises an Fc domain.
35B. The method of any one of Embodiments 1B, 2B, 6B, 10B, and 12B-34B, wherein the capture antibody, or the detection antibody, and/or the CAR comprises a VH and a VL, wherein:
   (i) the VH comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and
   (ii) the VL comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6.
36B. The method of Embodiment 35B, wherein the capture antibody, the detection antibody, or the CAR comprises a VH comprising the amino acid sequence of SEQ ID NO:7 or 8 and/or a VL comprising the amino acid sequence of SEQ ID NO:9 or 10.
37B. The method of Embodiment 36B, wherein the VH comprises the amino acid sequence of SEQ ID NO:7 and the VL comprises the amino acid sequence of SEQ ID NO:10.
38B. The method of any one of Embodiments 1B-37B, further comprising:
   a) detecting the level of one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in the biological sample;
   b) comparing the level of the one or more proteins to a reference level; and
   c) selecting the individual as having an autoimmune or inflammatory disease when the level of the one or more proteins in the biological sample is above the reference level.
39B. The method of Embodiment 6B, wherein the administering comprises:
   (a) isolating T cells from a biological sample obtained from the individual;
   (b) enriching the T cells for Tregs;
   (c) transfecting the enriched Treg cells with an expression vector comprising an expression control sequence operatively linked to a nucleotide sequence encoding a chimeric antigen receptor (CAR) comprising an antigen binding domain that binds to a citrullinated polypeptide or citrullinated peptide;
   (d) expanding the transfected Treg cells; and
   (e) administering a composition comprising the expanded Treg cells to the individual.
40B. The method of any one of Embodiments 1B-39B, wherein the individual is a human.
41B. The method of any one of Embodiments 6B, 39B, and 40B, wherein the composition comprising the expanded Treg cells is administered intravenously.
42B. The method of any one of Embodiments 3B, 4B, 7B-9B, and 12B-41B, wherein the CAR comprises an optional signal peptide, a CAR antigen binding domain that specifically binds to a citrullinated polypeptide or citrullinated peptide, an optional hinge domain, a transmembrane domain, one or more optional co-stimulatory domains, and an intracellular signaling domain.
43B. The method of Embodiment 42B, wherein the CAR antigen binding domain binds to one or more of: citrullinated vimentin, citrullinated filaggrin, citrullinated fibrinogen, citrullinated tenascin C, or citrullinated fragments thereof.
44B. The method of Embodiment 42B or Embodiment 43B, wherein the CAR antigen binding domain comprises an antibody fragment selected from the group consisting of an Fv, scFv, camelid nanobody, heavy chain only antibody, or aptamer.
45B. The method of any one of Embodiments 42B-44B, wherein the CAR antigen binding domain comprises an scFv.
46B. The method of any one of Embodiments 42B-45B, wherein the CAR antigen binding domain comprises the same VH and VL as the capture antibody or detection antibody of any one of Embodiments 1-37.
47B. The method of Embodiment 45B, wherein antigen binding domain of the CAR comprises an scFV comprising a VH and a VL joined by a linker selected from the group consisting of SEQ ID NOs:11-13.
48B. The method of Embodiment 47B, wherein the CAR antigen binding domain comprises the amino acid sequence of any one of SEQ ID NOs:14-16.
49B. The method of any one of Embodiments 42B-48B, wherein the intracellular signaling domain is derived from CD3-zeta or an Fc receptor.
50B. The method of any one of Embodiments 42B-49B, wherein the CAR comprises at least one co-stimulatory domain derived from the group consisting of: CD3-zeta, FceR1g, Fcg, CD28, 4-1BB, OX40, CD40L, MyD88, CD40, ICOS, RANK/TRANCE-R, CTLA-4, CTLA-4/CD-28 hybrid, DAP10, CD27, and 2B4.
51B. The method of any one of Embodiments 42B-50B, wherein the CAR further comprises a hinge domain.
52B. A kit comprising a capture antibody and a detection antibody and instructions for use according to any one of Embodiments 1B-51B.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

### Sequence Table

| | Sequence | SEQ ID NO: |
|---|---|---|
| CV VH-CDR1 | DTTYYWG | 1 |
| CV VH-CDR2 | SIYYRGNTHYNSSLRS | 2 |
| CV VH-CDR3 | LDPFDY | 3 |
| CV VL-CDR1 | GGNNIGSKSVH | 4 |
| CV VL-CDR2 | YDSDRPS | 5 |
| CV VL-CDR3 | QVWDSSSDHQV | 6 |
| CV VH #1 | HLHLQESGPGLVKPSETLSLTCTVSGGSINDTTYY | 7 |
| | WGWIRQPPGKGLEWIGSIYYRGNTHYNSSLRSRV | |
| | TMSVDTSKNRFSLKVTSVTAADTAVYYCARLDPF | |
| | DYWGRGTLVTVSS | |
| CV VH #2 | QLQLQESGPGLVKPSETLSLTCTVSGGSISSSSYY | 8 |
| | WGWIRQPPGKGLEWIGSIYYSGSTYYNPSLKSRV | |
| | TISVDTSKNQFSLKLSSVTAADTAVYYCARLDPFD | |
| | YWGRGTLVTVSS | |
| CV VL1 | SYVLTQPPSVSLAPGETATITCGGDDIENQNVNW | 9 |
| | YQQKSGQAPMLLIFFDTRRPSGIPERFSGSRSEDTA | |
| | NLTITRVEAGDDADYFCQVYDRKTDHQVFGPGTT | |
| | VTVL | |
| CV VL2 | SYVLTQPPSVSVAPGKTARITCGGNNIGSKSVHW | 10 |
| | YQQKPGQAPVLVIYYDSDRPSGIPERFSGSNSGNT | |
| | ATLTISRVEAGDEADYYCQVWDSSSDHQVFGTGT | |
| | KVTVL | |
| Linker 1 | GGGGSGGGGSGGGGS | 11 |
| Linker 2/Whitlow | GSTSGSGKPGSGEGSTKG | 12 |
| 218 Linker | | |
| Linker 3/AB pur | ASSGGSTSGSGKPGSGEGSSGSAR | 13 |
| Linker | | |
| CV VH-(L)-VL #1 | HLHLQESGPGLVKPSETLSLTCTVSGGSINDTTYY | 14 |
| | WGWIRQPPGKGLEWIGSIYYRGNTHYNSSLRSRV | |
| | TMSVDTSKNRFSLKVTSVTAADTAVYYCARLDPF | |
| | DYWGRGTLVTVSSGGGGSGGGGSGGGGSSYVL | |
| | TOPPSVSVAPGKTARITCGGNNIGSKSVHWYQQK | |
| | PGQAPVLVIYYDSDRPSGIPERFSGSNSGNTATLTI | |
| | SRVEAGDEADYYCQVWDSSSDHQVFGTGTKVTV | |
| | LR | |
| CV VH-(L)-VL #2 | HLHLQESGPGLVKPSETLSLTCTVSGGSINDTTYY | 15 |
| | WGWIRQPPGKGLEWIGSIYYRGNTHYNSSLRSRV | |
| | TMSVDTSKNRFSLKVTSVTAADTAVYYCARLDPF | |
| | DYWGRGTLVTVSSGSTSGSGKPGSGEGSTKGSY | |
| | VLTQPPSVSVAPGKTARITCGGNNIGSKSVHWYQ | |
| | QKPGQAPVLVIYYDSDRPSGIPERFSGSNSGNTAT | |
| | LTISRVEAGDEADYYCQVWDSSSDHQVFGTGTK | |
| | VTVLR | |
| CV VH-(L)-VL #3 | HLHLQESGPGLVKPSETLSLTCTVSGGSINDTTYY | 16 |
| | WGWIRQPPGKGLEWIGSIYYRGNTHYNSSLRSRV | |
| | TMSVDTSKNRFSLKVTSVTAADTAVYYCARLDPF | |
| | DYWGRGTLVTVSSASSGGSTSGSGKPGSGEGSS | |
| | GSARSYVLTQPPSVSVAPGKTARITCGGNNIGSKS | |
| | VHWYQQKPGQAPVLVIYYDSDRPSGIPERFSGSN | |
| | SGNTATLTISRVEAGDEADYYCQVWDSSSDHQVF | |
| | GTGTKVTVLR | |
| CV VH #1 | CACCTGCACTTGCAGGAGTCGGGCCCAGGACTT | 17 |
| nucleic acid | GTGAAGCCTTCGGAGACCCTGTCCCTCACCTGC | |
| | ACTGTCTCTGGTGGCTCCATCAACGATACCACT | |
| | TACTACTGGGGCTGGATTCGCCAGCCCCCCGGG | |
| | AAGGGACTGGAGTGGATTGGGAGTATCTATTAC | |
| | CGGGGGAACACCCACTACAATTCGTCCCTGAGG | |
| | AGTCGCGTCACCATGTCTGTCGACACTTCCAAG | |
| | AACCGATTCTCCCTGAAGGTCACTTCTGTGACT | |
| | GCCGCAGACACGGCTGTCTATTACTGTGCGAGA | |
| | CTCGACCCATTTGACTACTGGGGCCGTGGCACC | |
| | CTGGTCACTGTCTCGAGC | |
| CV VH #2 | CAGCTGCAGCTGCAGGAGTCGGGCCCAGGACT | 18 |
| nucleic acid | GGTGAAGCCTTCGGAGACCCTGTCCCTCACCTG | |
| | CACTGTCTCTGGTGGCTCCATCAGCAGTAGTAG | |
| | TTACTACTGGGGCTGGATCCGCCAGCCCCCAGG | |
| | GAAGGGGCTGGAGTGGATTGGGAGTATCTATTA | |
| | TAGTGGGAGCACCTACTACAACCCGTCCCTCAA | |
| | GAGTCGAGTCACCATATCCGTAGACACGTCCAA | |
| | GAACCAGTTCTCCCTGAAGCTGAGCTCTGTGAC | |
| | CGCCGCAGACACGGCTGTGTATTACTGTGCGAG | |
| | ACTCGACCCATTTGACTACTGGGGCCGTGGCAC | |
| | CCTGGTCACTGTCTCGAGC | |
| CV VL #1 | TCCTATGTCCTGACTCAGCCACCCTCAGTGTCG | 19 |
| nucleic acid | CTGGCCCCGGGAGAGACGGCCACAATTACTTGT | |
| | GGTGGAGACGACATTGAAAATCAAAATGTCAA | |
| | CTGGTATCAGCAGAAGTCAGGTCAGGCCCCTAT | |
| | GCTGCTCATCTTCTTTGATACCAGACGGCCCTC | |
| | AGGGATCCCGGAGCGATTCTCTGGCTCCAGGTC | |
| | TGAGGACACGGCCAACCTGACCATCACCAGGG | |
| | TCGAGGCCGGGGATGACGCCGACTATTTCTGTC | |
| | AGGTGTATGATAGGAAGACTGATCACCAAGTCT | |
| | TCGGACCTGGGACCACGGTCACCGTCCTA | |
| CV VL #2 | TCCTATGTGCTGACTCAGCCACCCTCAGTGTCA | 20 |
| nucleic acid | GTGGCCCCAGGAAAGACGGCCAGGATTACCTG | |
| | TGGGGGAAACAACATTGGAAGTAAAAGTGTGC | |
| | ACTGGTACCAGCAGAAGCCAGGCCAGGCCCCT | |
| | GTGCTGGTCATCTATTATGATAGCGACCGGCCC | |
| | TCAGGGATCCCTGAGCGATTCTCTGGCTCCAAC | |
| | TCTGGGAACACGGCCACCCTGACCATCAGCAGG | |
| | GTCGAAGCCGGGGATGAGGCCGACTATTACTGT | |
| | CAGGTGTGGGACAGTAGTAGTGATCACCAAGTC | |
| | TTCGGAACTGGGACCAAGGTCACCGTCCTA | |
| Linker 1 | GGTGGCGGTGGCTCGGGCGGTGGTGGGTCGGG | 21 |
| nucleic acid | TGGCGGCGGATCT | |
| Linker 2/Whitlow | GGAAGCACGAGTGGTTCAGGCAAACCGGGTTC | 22 |
| 218 Linker | CGGTGAAGGTTCAACAAAAGGT | |
| nucleic acid | | |
| Linker 3/AB pur | GCCTCTAGCGGGGGGAGCACATCAGGAAGCGG | 23 |
| Linker | CAAGCCCGGTAGCGGCGAAGGCTCCTCTGGCA | |
| nucleic acid | GCGCCCGC | |
| CV VH-(L)-VL #1 | CACCTGCACTTGCAGGAGTCGGGCCCAGGACTT | 24 |
| nucleic acid | GTGAAGCCTTCGGAGACCCTGTCCCTCACCTGC | |
| | ACTGTCTCTGGTGGCTCCATCAACGATACCACT | |
| | TACTACTGGGGCTGGATTCGCCAGCCCCCCGGG | |
| | AAGGGACTGGAGTGGATTGGGAGTATCTATTAC | |
| | CGGGGGAACACCCACTACAATTCGTCCCTGAGG | |
| | AGTCGCGTCACCATGTCTGTCGACACTTCCAAG | |
| | AACCGATTCTCCCTGAAGGTCACTTCTGTGACT | |
| | GCCGCAGACACGGCTGTCTATTACTGTGCGAGA | |
| | CTCGACCCATTTGACTACTGGGGCCGTGGCACC | |
| | CTGGTCACTGTCTCGAGCGGTGGCGGTGGCTCG | |
| | GGCGGTGGTGGGTCGGGTGGCGGCGGATCTTCC | |
| | TATGTGCTGACTCAGCCACCCTCAGTGTCAGTG | |
| | GCCCCAGGAAAGACGGCCAGGATTACCTGTGG | |
| | GGGAAACAACATTGGAAGTAAAAGTGTGCACT | |
| | GGTACCAGCAGAAGCCAGGCCAGGCCCCTGTG | |
| | CTGGTCATCTATTATGATAGCGACCGGCCCTCA | |
| | GGGATCCCTGAGCGATTCTCTGGCTCCAACTCT | |
| | GGGAACACGGCCACCCTGACCATCAGCAGGGT | |
| | CGAAGCCGGGGATGAGGCCGACTATTACTGTCA | |
| | GGTGTGGGACAGTAGTAGTGATCACCAAGTCTT | |
| | CGGAACTGGGACCAAGGTCACCGTCCTACGC | |
| CV VH-(L)-VL #2 | CACCTGCACTTGCAGGAGTCGGGCCCAGGACTT | 25 |
| nucleic acid | GTGAAGCCTTCGGAGACCCTGTCCCTCACCTGC | |
| | ACTGTCTCTGGTGGCTCCATCAACGATACCACT | |
| | TACTACTGGGGCTGGATTCGCCAGCCCCCCGGG | |
| | AAGGGACTGGAGTGGATTGGGAGTATCTATTAC | |
| | CGGGGGAACACCCACTACAATTCGTCCCTGAGG | |
| | AGTCGCGTCACCATGTCTGTCGACACTTCCAAG | |
| | AACCGATTCTCCCTGAAGGTCACTTCTGTGACT | |
| | GCCGCAGACACGGCTGTCTATTACTGTGCGAGA | |
| | CTCGACCCATTTGACTACTGGGGCCGTGGCACC | |
| | CTGGTCACTGTCTCGAGCGGAAGCACGAGTGGT | |
| | TCAGGCAAACCGGGTTCCGGTGAAGGTTCAACA | |
| | AAAGGTTCCTATGTGCTGACTCAGCCACCCTCA | |
| | GTGTCAGTGGCCCCAGGAAAGACGGCCAGGAT | |
| | TACCTGTGGGGGAAACAACATTGGAAGTAAAA | |
| | GTGTGCACTGGTACCAGCAGAAGCCAGGCCAG | |
| | GCCCCTGTGCTGGTCATCTATTATGATAGCGAC | |
| | CGGCCCTCAGGGATCCCTGAGCGATTCTCTGGC | |
| | TCCAACTCTGGGAACACGGCCACCCTGACCATC | |
| | AGCAGGGTCGAAGCCGGGGATGAGGCCGACTA | |
| | TTACTGTCAGGTGTGGGACAGTAGTAGTGATCA | |
| | CCAAGTCTTCGGAACTGGGACCAAGGTCACCGT | |
| | CCTACGC | |
| CV VH-(L)-VL #3 | CACCTGCACTTGCAGGAGTCGGGCCCAGGACTT | 26 |
| nucleic acid | GTGAAGCCTTCGGAGACCCTGTCCCTCACCTGC | |
| | ACTGTCTCTGGTGGCTCCATCAACGATACCACT | |
| | TACTACTGGGGCTGGATTCGCCAGCCCCCCGGG | |
| | AAGGGACTGGAGTGGATTGGGAGTATCTATTAC | |
| | CGGGGGAACACCCACTACAATTCGTCCCTGAGG | |
| | AGTCGCGTCACCATGTCTGTCGACACTTCCAAG | |
| | AACCGATTCTCCCTGAAGGTCACTTCTGTGACT | |
| | GCCGCAGACACGGCTGTCTATTACTGTGCGAGA | |
| | CTCGACCCATTTGACTACTGGGGCCGTGGCACC | |
| | CTGGTCACTGTCTCGAGCGCCTCTAGCGGGGGG | |
| | AGCACATCAGGAAGCGGCAAGCCCGGTAGCGG | |
| | CGAAGGCTCCTCTGGCAGCGCCCGCTCCTATGT | |
| | GCTGACTCAGCCACCCTCAGTGTCAGTGGCCCC | |
| | AGGAAAGACGGCCAGGATTACCTGTGGGGGAA | |
| | ACAACATTGGAAGTAAAAGTGTGCACTGGTACC | |
| | AGCAGAAGCCAGGCCAGGCCCCTGTGCTGGTC | |
| | ATCTATTATGATAGCGACCGGCCCTCAGGGATC | |
| | CCTGAGCGATTCTCTGGCTCCAACTCTGGGAAC | |
| | ACGGCCACCCTGACCATCAGCAGGGTCGAAGC | |
| | CGGGGATGAGGCCGACTATTACTGTCAGGTGTG | |
| | GGACAGTAGTAGTGATCACCAAGTCTTCGGAAC | |
| | TGGGACCAAGGTCACCGTCCTACGC | |
| Citrullinated | ST(Cit)SVSSSSY(Cit)(Cit)MFGG | 27 |
| antigen domain #1 | | |
| Citrullinated | VYAT(Cit)SSAV(Cit)L(Cit)SSV | 28 |
| antigen domain #2 | | |
| Citrullinated | (Cit)PAPPPISGGGY(Cit)A(Cit) | 29 |
| antigen domain #3 | | |
| Citrullinated | SHQEST(Cit)GRSRGRSGRSGS | 30 |
| antigen domain #4 | | |
| HFR1 | HLHLQESGPGLVKPSETLSLTCTVSGGSIN | 31 |
| HFR2 | WIRQPPGKGLEWIG | 32 |
| HFR3 | RVTMSVDTSKNRFSLKVTSVTAADTAVYYCAR | 33 |
| HER4 | WGRGTLVTVSS | 34 |
| LFR1 | SYVLTQPPSVSVAPGKTARITC | 35 |
| LFR2 | WYQQKPGQAPVLVIY | 36 |
| LFR3 | GIPERFSGSNSGNTATLTISRVEAGDEADYYC | 37 |
| LFR4 | FGTGTKVTV | 38 |
| Signal peptide | MALPVTALLLPLALLLHAAR | 39 |
| CD28 hinge | IEVMYPPPYLDNEKSNGTIIHVKGKHLCPSPLFPGP | 40 |
| | SKP | |
| CD8 hinge | TTTPAPRPPTPAPTIASQPLSLRPEACRPAAGGAVH | 41 |
| | TRGLDFACDIYIWAPLAGTCGVLLLSLVITLYC | |
| Transmembrane | FWVLVVVGGVLACYSLLVTVAFIIFWV | 42 |
| domain #1 | | |
| Transmembrane | IYIWAPLAGTCGVLLLSLVITLYC | 43 |
| domain #2 | | |
| CD3ζ signal | RVKFSRSADAPAYQQGQNQLYNELNLGRREEYD | 44 |
| transduction | VLDKRRGRDPEMGGKPRRKNPQEGLYNELQKDK | |
| domain | MAEAYSEIGMKGERRRGKGHDGLYQGLSTATKD | |
| | TYDALHMQALPPR | |
| CD28 co- | RSKRSRLLHSDYMNMTPRRPGPTRKHYQPYAPPR | 45 |
| stimulatory | DFAAYRS | |
| domain | | |
| 4-1BB co- | KRGRKKLLYIFKQPFMRPVQTTQEEDGCSCRFPEE | 46 |
| stimulatory | EEGGCEL | |
| domain | | |

What is claimed:

1. A method of detecting the presence or amount of a citrullinated polypeptide or citrullinated peptide in a biological sample obtained from an individual comprising
a) incubating the biological sample with a capture antibody that binds to the citrullinated polypeptide or the citrullinated peptide to form a complex comprising the capture antibody and the citrullinated polypeptide or citrullinated peptide;
b) incubating the complex of the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
c) detecting the complex comprising the capture antibody, the citrullinated polypeptide or the citrullinated peptide, and the detection antibody, thereby detecting the presence or amount of the citrullinated polypeptide or citrullinated peptide.

2. A method of diagnosing an autoimmune or inflammatory disease in an individual comprising
a) incubating a biological sample obtained from the individual with a capture antibody that binds to a citrullinated polypeptide or citrullinated peptide to form a complex comprising the capture antibody and the citrullinated polypeptide or citrullinated peptide;
b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
c) detecting the complex comprising the capture antibody, the citrullinated polypeptide or the citrullinated peptide, and the detection antibody to determine the presence or amount of the citrullinated polypeptide or citrullinated peptide, wherein the individual is diagnosed with the autoimmune or inflammatory disease based upon the presence or amount of the citrullinated polypeptide or citrullinated peptide.

3. A method of selecting an individual having an autoimmune or inflammatory disease for treatment with a T cell therapy comprising a T cell comprising a chimeric antigen receptor (CAR) that binds to a citrullinated polypeptide or citrullinated peptide comprising
a) detecting the presence or amount of the citrullinated polypeptide or citrullinated peptide in a biological sample obtained from the individual; and
b) selecting the individual for treatment with the T cell therapy if the biological sample comprises citrullinated polypeptides or citrullinated peptides or an amount of citrullinated polypeptides or citrullinated peptides that is above a threshold or a reference level.

4. The method of claim 3, further comprising comparing the amount of the citrullinated polypeptide or citrullinated peptide in the biological sample to a reference level or reference sample.

5. The method of claim 4, wherein the reference sample is a biological sample obtained from an individual not having an autoimmune or inflammatory disease.

6. A method of treating an autoimmune or inflammatory disease in an individual comprising
i) detecting a citrullinated polypeptide or citrullinated peptide in a biological sample obtained from an individual comprising
a) incubating the biological sample with a capture antibody that binds to a citrullinated polypeptide or citrullinated peptide to form a complex comprising the capture antibody and the citrullinated polypeptide or citrullinated peptide;
b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
c) detecting the complex comprising the capture antibody, the citrullinated polypeptide or the citrullinated peptide, and the detection antibody; and
ii) administering to the individual an effective amount of a composition comprising a T cell comprising a chimeric antigen receptor (CAR) comprising an antigen binding domain that specifically binds to a citrullinated polypeptide or citrullinated peptide, if the biological sample comprises citrullinated polypeptides or citrullinated peptides or an amount of citrullinated polypeptides or citrullinated peptides that is above a threshold or a reference level.

7. A method of monitoring treatment of an autoimmune or inflammatory disease in an individual comprising
a) detecting the presence or amount of a citrullinated polypeptide or citrullinated peptide in a biological sample obtained from the individual receiving treatment for the autoimmune or inflammatory disease; and
b) comparing the presence or amount of the citrullinated polypeptide or citrullinated peptide in the biological sample to a biological sample obtained from the individual prior to receiving treatment for the autoimmune or inflammatory disease or from an earlier time point during treatment,
wherein a reduction in the amount of citrullinated polypeptides or citrullinated peptides in a biological sample compared to the amount of citrullinated polypeptides or citrullinated peptides in a biological sample prior to receiving treatment for the autoimmune or inflammatory disease or from an earlier time point during treatment is indicative of the effective treatment for the autoimmune or inflammatory disease in the individual.

8. A method of diagnosing an autoimmune or inflammatory disease in an individual comprising
a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
b) detecting the signal emitted by the CAR to determine the presence or amount of the citrullinated polypeptide or citrullinated peptide, wherein the individual is diagnosed with rheumatoid arthritis based upon the presence or amount of the citrullinated polypeptide or citrullinated peptide.

9. A method of treating an autoimmune or inflammatory disease in an individual comprising
a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated;
b) detecting the signal emitted by the CAR to determine the presence or amount of the citrullinated polypeptide or citrullinated peptide; and
c) administering to the individual an effective amount of a composition comprising a T cell comprising a chimeric antigen receptor (CAR) comprising an antigen binding domain that specifically binds to a citrullinated polypeptide or citrullinated peptide if the biological sample comprises citrullinated polypeptides or citrullinated peptides or an amount of citrullinated polypeptides or citrullinated peptides that is above a threshold or a reference level.

10. The method of claim 3 or claim 7, wherein the detecting comprises
a) incubating the biological sample with a capture antibody that binds to the citrullinated polypeptide or citrullinated peptide to form a complex comprising the capture antibody and the citrullinated polypeptide or citrullinated peptide;
b) incubating the complex comprising the capture antibody and the citrullinated polypeptide or citrullinated peptide with a detection antibody that binds to the citrullinated polypeptide or citrullinated peptide to form a complex comprising the capture antibody, the citrullinated polypeptide or the citrullinated peptide, and the detection antibody; and
c) detecting the complex comprising the capture antibody, the citrullinated polypeptide or the citrullinated peptide, and the detection antibody.

11. The method of claim 3 or claim 7, wherein the detecting comprises:
a) incubating a biological sample obtained from the individual with a cell comprising a chimeric antigen receptor (CAR) that binds to a citrullinated polypeptide or citrullinated peptide to form a complex between the CAR and the citrullinated polypeptide or citrullinated peptide, wherein the cell emits a signal when the CAR is activated; and
b) detecting the signal emitted by the CAR to determine the presence or amount of the citrullinated polypeptide or citrullinated peptide.

12. The method of any one of claims 2-11, wherein the autoimmune or inflammatory disease is selected from the group consisting of rheumatoid arthritis, hidradenitis suppurativa, rheumatoid arthritis-associated interstitial lung disease, multiple sclerosis, Alzheimer's disease, systematic lupus erythematosus, polymyositis, inflammatory bowel disease, chronic tonsillitis, type 1 autoimmune hepatitis, psoriatic arthritis, systemic sclerosis, pemphigus vulgaris, pemphigus foliaceus, aggressive periodontitis, and autoimmune encephalitis associated with prion disease.

13. The method of any one of claims 1-12, wherein the biological sample is selected from the group consisting of cerebrospinal fluid, spinal cord white matter, lesional skin biopsy, sweat, saliva, pus, mucus, bile, liver biopsy, brain biopsy, pleural fluid, peritoneal fluid, blood, blood plasma, serum, synovial fluid, and any combination thereof.

14. The method of any one of claims 1-13, wherein the biological sample is serum.

15. The method of any one of claims 1-13, wherein the biological sample is synovial fluid.

16. The method of claim 13, wherein the biological sample is serum and synovial fluid, and wherein the serum and the synovial fluid are from the same individual.

17. The method of any one of claims 1, 2, 6, 10, and 12-16, wherein the capture antibody is immobilized to a sample plate.

18. The method of any one of claims 1, 2, 6, 10, and 12-16, wherein the capture antibody is immobilized to a bead.

19. The method of any one of claims 1, 2, 6, 10, and 12-18, wherein the detection antibody is conjugated to a label molecule.

20. The method of any one of claims 1, 2, 6, 10, and 12-19, wherein the detection antibody is detected by the binding of a secondary antibody, wherein the secondary antibody is conjugated to a label molecule.

21. The method of claim 20, wherein the secondary antibody binds to a constant region of the detection antibody.

22. The method of claim 21, wherein the secondary antibody is an anti-IgM antibody or fragment thereof.

23. The method of any one of claims 19-22, wherein the label molecule is selected from the group consisting of a chemiluminescent enzyme, a fluorescent tag, and a radiolabeled tag.

24. The method of any one of claims 19-23, wherein the label molecule is a horseradish peroxidase enzyme or an alkaline phosphatase enzyme.

25. The method of claim 24, wherein the horseradish peroxidase enzyme or alkaline phosphatase enzyme bind a substrate molecule, wherein the substrate molecule is converted into a detectable product.

26. The method of claim 25, wherein the substrate molecule is colorimetric/chromogenic, fluorescent, chemifluorescent, or chemiluminescent.

27. The method of any one of claims 1, 2, 6, 10, and 12-26, wherein the capture antibody and the detection antibody bind to different epitopes of the citrullinated polypeptide or citrullinated peptide.

28. The method of claim 27, wherein the capture antibody and/or the detection antibody binds to one or more of: citrullinated vimentin, citrullinated filaggrin, citrullinated fibrinogen, citrullinated tenascin C, and/or citrullinated fragments thereof.

29. The method of claim 28, wherein the capture antibody and/or the detection antibody binds to two or more of citrullinated vimentin, citrullinated filaggrin, citrullinated fibrinogen, citrullinated tenascin C, and/or citrullinated fragments thereof.

30. The method of claim 28 or claim 29, wherein the capture antibody or the detection antibody is an anti-peptidyl-citrulline antibody.

31. The method of claim 30, wherein the anti-peptidyl-citrulline antibody is the F95 antibody clone.

32. The method of any one of claims 1, 2, 6, 10, and 12-31, wherein the capture antibody or the detection antibody comprises a full-length antibody or an antibody fragment selected from the group consisting of a Fv, scFv, camelid nanobody, heavy chain only antibody, and aptamer.

33. The method of claim 32, wherein the capture antibody or the detection antibody comprises a full-length antibody.

34. The method of claim 33, wherein the capture antibody comprises an Fc domain.

35. The method of any one of claims 1, 2, 6, 10, and 12-34, wherein the capture antibody, or the detection antibody, and/or the CAR comprises a VH and a VL, wherein:
(i) the VH comprises a VH-CDR1 comprising the amino acid sequence of SEQ ID NO:1, a VH-CDR2 comprising the amino acid sequence of SEQ ID NO:2, and a VH-CDR3 comprising the amino acid sequence of SEQ ID NO:3, and
(ii) the VL comprises a VL-CDR1 comprising the amino acid sequence of SEQ ID NO:4, a VL-CDR2 comprising the amino acid sequence of SEQ ID NO:5, and a VL-CDR3 comprising the amino acid sequence of SEQ ID NO:6.

36. The method of claim 35, wherein the capture antibody, the detection antibody, or the CAR comprises a VH comprising the amino acid sequence of SEQ ID NO:7 or 8 and/or a VL comprising the amino acid sequence of SEQ ID NO:9 or 10.

37. The method of claim 36, wherein the VH comprises the amino acid sequence of SEQ ID NO:7 and the VL comprises the amino acid sequence of SEQ ID NO:10.

38. The method of any one of claims 1-37, further comprising:
a) detecting the level of one or more proteins selected from the group consisting of C reactive protein (CRP), IL-1β, IL-6, IL-8, IL-17, IL-23, IL-18, IL-12p70, IP-10, MCP-1, MIP-1a, IFN-γ, and TNF-α in the biological sample;
b) comparing the level of the one or more proteins to a reference level; and
c) selecting the individual as having an autoimmune or inflammatory disease when the level of the one or more proteins in the biological sample is above the reference level.

39. The method of claim 6, wherein the administering comprises:
(a) isolating T cells from a biological sample obtained from the individual;
(b) enriching the T cells for Tregs;
(c) transfecting the enriched Treg cells with an expression vector comprising an expression control sequence operatively linked to a nucleotide sequence encoding a chimeric antigen receptor (CAR) comprising an antigen binding domain that binds to a citrullinated polypeptide or citrullinated peptide;
(d) expanding the transfected Treg cells; and
(e) administering a composition comprising the expanded Treg cells to the individual.

40. The method of any one of claims 1-39, wherein the individual is a human.

41. The method of any one of claims 6, 39, and 40, wherein the composition comprising the expanded Treg cells is administered intravenously.

42. The method of any one of claims 3, 4, 7-9, and 12-41, wherein the CAR comprises an optional signal peptide, a CAR antigen binding domain that specifically binds to a citrullinated polypeptide or citrullinated peptide, an optional hinge domain, a transmembrane domain, one or more optional co-stimulatory domains, and an intracellular signaling domain.

43. The method of claim 42, wherein the CAR antigen binding domain binds to one or more of: citrullinated vimentin, citrullinated filaggrin, citrullinated fibrinogen, citrullinated tenascin C, or citrullinated fragments thereof.

44. The method of claim 42 or claim 43, wherein the CAR antigen binding domain comprises an antibody fragment selected from the group consisting of an Fv, scFv, camelid nanobody, heavy chain only antibody, or aptamer.

45. The method of any one of claims 42-44, wherein the CAR antigen binding domain comprises an scFv.

46. The method of any one of claims 42-45, wherein the CAR antigen binding domain comprises the same VH and VL as the capture antibody or detection antibody of any one of claims 1-37.

47. The method of claim 45, wherein antigen binding domain of the CAR comprises an scFV comprising a VH and a VL joined by a linker selected from the group consisting of SEQ ID NOs:11-13.

48. The method of claim 47, wherein the CAR antigen binding domain comprises the amino acid sequence of any one of SEQ ID NOs:14-16.

49. The method of any one of claims 42-48, wherein the intracellular signaling domain is derived from CD3-zeta or an Fc receptor.

50. The method of any one of claims 42-49, wherein the CAR comprises at least one co-stimulatory domain derived from the group consisting of: CD3-zeta, FceR1g, Fcg, CD28, 4-1BB, OX40, CD40L, MyD88, CD40, ICOS, RANK/TRANCE-R, CTLA-4, CTLA-4/CD-28 hybrid, DAP10, CD27, and 2B4.

51. The method of any one of claims 42-50, wherein the CAR further comprises a hinge domain.

52. A kit comprising a capture antibody and a detection antibody and instructions for use according to any one of claims 1-51.


SEQUENCE LISTING (46 sequences)

SEQ ID NO: 1 - protein, 7 aa, synthetic construct
DTTYYWG

SEQ ID NO: 2 - protein, 16 aa, synthetic construct
SIYYRGNTHYNSSLRS

SEQ ID NO: 3 - protein, 6 aa, synthetic construct
LDPFDY

SEQ ID NO: 4 - protein, 11 aa, synthetic construct
GGNNIGSKSVH

SEQ ID NO: 5 - protein, 7 aa, synthetic construct
YDSDRPS

SEQ ID NO: 6 - protein, 11 aa, synthetic construct
QVWDSSSDHQV

SEQ ID NO: 7 - protein, 116 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSS

SEQ ID NO: 8 - protein, 116 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARLDPFDYWGRGTLVTVSS

SEQ ID NO: 9 - protein, 108 aa, synthetic construct
SYVLTQPPSVSLAPGETATITCGGDDIENQNVNWYQQKSGQAPMLLIFFDTRRPSGIPERFSGSRSEDTANLTITRVEAGDDADYFCQVYDRKTDHQVFGPGTTVTVL

SEQ ID NO: 10 - protein, 108 aa, synthetic construct
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVL

SEQ ID NO: 11 - protein, 15 aa, synthetic construct
GGGGSGGGGSGGGGS

SEQ ID NO: 12 - protein, 18 aa, synthetic construct
GSTSGSGKPGSGEGSTKG

SEQ ID NO: 13 - protein, 24 aa, synthetic construct
ASSGGSTSGSGKPGSGEGSSGSAR

SEQ ID NO: 14 - protein, 240 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSGGGGSGGGGSGGGGSSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 15 - protein, 243 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSGSTSGSGKPGSGEGSTKGSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 16 - protein, 249 aa, synthetic construct
HLHLQESGPGLVKPSETLSLTCTVSGGSINDTTYYWGWIRQPPGKGLEWIGSIYYRGNTHYNSSLRSRVTMSVDTSKNRFSLKVTSVTAADTAVYYCARLDPFDYWGRGTLVTVSSASSGGSTSGSGKPGSGEGSSGSARSYVLTQPPSVSVAPGKTARITCGGNNIGSKSVHWYQQKPGQAPVLVIYYDSDRPSGIPERFSGSNSGNTATLTISRVEAGDEADYYCQVWDSSSDHQVFGTGTKVTVLR

SEQ ID NO: 17 - DNA, 348 bp, synthetic construct
  source 1..348: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagc

SEQ ID NO: 18 - DNA, 348 bp, synthetic construct
  source 1..348: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagactcgacccatttgactactggggccgtggcaccctggtcactgtctcgagc

SEQ ID NO: 19 - DNA, 324 bp, synthetic construct
  source 1..324: mol_type = other DNA
tcctatgtcctgactcagccaccctcagtgtcgctggccccgggagagacggccacaattacttgtggtggagacgacattgaaaatcaaaatgtcaactggtatcagcagaagtcaggtcaggcccctatgctgctcatcttctttgataccagacggccctcagggatcccggagcgattctctggctccaggtctgaggacacggccaacctgaccatcaccagggtcgaggccggggatgacgccgactatttctgtcaggtgtatgataggaagactgatcaccaagtcttcggacctgggaccacggtcaccgtccta

SEQ ID NO: 20 - DNA, 324 bp, synthetic construct
  source 1..324: mol_type = other DNA
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtccta

SEQ ID NO: 21 - DNA, 45 bp, synthetic construct
  source 1..45: mol_type = other DNA
ggtggcggtggctcgggcggtggtgggtcgggtggcggcggatct

SEQ ID NO: 22 - DNA, 54 bp, synthetic construct
  source 1..54: mol_type = other DNA
ggaagcacgagtggttcaggcaaaccgggttccggtgaaggttcaacaaaaggt

SEQ ID NO: 23 - DNA, 72 bp, synthetic construct
  source 1..72: mol_type = other DNA
gcctctagcggggggagcacatcaggaagcggcaagcccggtagcggcgaaggctcctctggcagcgcccgc

SEQ ID NO: 24 - DNA, 720 bp, synthetic construct
  source 1..720: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcggtggcggtggctcgggcggtggtgggtcgggtggcggcggatcttcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 25 - DNA, 729 bp, synthetic construct
  source 1..729: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcggaagcacgagtggttcaggcaaaccgggttccggtgaaggttcaacaaaaggttcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 26 - DNA, 747 bp, synthetic construct
  source 1..747: mol_type = other DNA
cacctgcacttgcaggagtcgggcccaggacttgtgaagccttcggagaccctgtccctcacctgcactgtctctggtggctccatcaacgataccacttactactggggctggattcgccagccccccgggaagggactggagtggattgggagtatctattaccgggggaacacccactacaattcgtccctgaggagtcgcgtcaccatgtctgtcgacacttccaagaaccgattctccctgaaggtcacttctgtgactgccgcagacacggctgtctattactgtgcgagactcgacccatttgactactggggccgtggcaccctggtcactgtctcgagcgcctctagcggggggagcacatcaggaagcggcaagcccggtagcggcgaaggctcctctggcagcgcccgctcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcaccaagtcttcggaactgggaccaaggtcaccgtcctacgc

SEQ ID NO: 27 - protein, 16 aa, synthetic construct
  VARIANT 3: note = X is citrulline
  VARIANT 11..12: note = X is citrulline
STXSVSSSSYXXMFGG

SEQ ID NO: 28 - protein, 15 aa, synthetic construct
  VARIANT 5: note = X is citrulline
  VARIANT 10: note = X is citrulline
  VARIANT 12: note = X is citrulline
VYATXSSAVXLXSSV

SEQ ID NO: 29 - protein, 15 aa, synthetic construct
  VARIANT 1: note = X is citrulline
  VARIANT 13: note = X is citrulline
  VARIANT 15: note = X is citrulline
XPAPPPISGGGYXAX

SEQ ID NO: 30 - protein, 19 aa, synthetic construct
  VARIANT 7: note = X is citrulline
SHQESTXGRSRGRSGRSGS

SEQ ID NO: 31 - protein, 30 aa, synthetic construct
HLHLQESGPGLVKPSETLSLTCTVSGGSIN

SEQ ID NO: 32 - protein, 14 aa, synthetic construct
WIRQPPGKGLEWIG

SEQ ID NO: 33 - protein, 32 aa, synthetic construct
RVTMSVDTSKNRFSLKVTSVTAADTAVYYCAR

SEQ ID NO: 34 - protein, 11 aa, synthetic construct
WGRGTLVTVSS

SEQ ID NO: 35 - protein, 22 aa, synthetic construct
SYVLTQPPSVSVAPGKTARITC

SEQ ID NO: 36 - protein, 15 aa, synthetic construct
WYQQKPGQAPVLVIY

SEQ ID NO: 37 - protein, 32 aa, synthetic construct
GIPERFSGSNSGNTATLTISRVEAGDEADYYC

SEQ ID NO: 38 - protein, 9 aa, synthetic construct
FGTGTKVTV

SEQ ID NO: 39 - protein, 20 aa, synthetic construct
MALPVTALLLPLALLLHAAR

SEQ ID NO: 40 - protein, 39 aa, synthetic construct
IEVMYPPPYLDNEKSNGTIIHVKGKHLCPSPLFPGPSKP

SEQ ID NO: 41 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 42 - protein, 27 aa, synthetic construct
FWVLVVVGGVLACYSLLVTVAFIIFWV

SEQ ID NO: 43 - protein, 24 aa, synthetic construct
IYIWAPLAGTCGVLLLSLVITLYC

SEQ ID NO: 44 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 41 aa, synthetic construct
RSKRSRLLHSDYMNMTPRRPGPTRKHYQPYAPPRDFAAYRS

SEQ ID NO: 46 - protein, 42 aa, synthetic construct
KRGRKKLLYIFKQPFMRPVQTTQEEDGCSCRFPEEEEGGCEL